(19) United States
(12) Patent Application Publication
(10) Pub. No.: US 2026/0060773 A1
(43) Pub. Date: Mar. 5, 2026

(54) Radiopaque Components and Method of Making the Same

(71) Applicant: ATEX Technologies, Inc., Pinebluff, NC (US)

(72) Inventors: Joseph M. SHEPLEY, Wernersville, PA (US); Aaron HJELLE, Andover, MN (US); Brian MCMURRAY, Aberdeen, NC (US)

(21) Appl. No.: 19/106,027

(22) PCT Filed: Aug. 24, 2023
(86) PCT No.: PCT/US2023/031028

§ 371 Date: Feb. 24, 2025

Related U.S. Application Data
(60) Provisional application No. 63/400,479, filed on Aug. 24, 2022.

(51) Int. Cl. A61B 90/00; A61K 49/04

(52) U.S. Cl. CPC A61B 90/39; A61K 49/0414; A61B 2090/3966

(57) ABSTRACT

A radiopaque component for an implantable device is provided herein. The radiopaque component comprises a plurality of radiopaque particles entrapped within a biostable and biocompatible binder. The radiopaque particles comprise at least 50% by weight of the radiopaque component.

### CROSS-REFERENCE TO RELATED APPLICATIONS

This application claims the priority benefit of U.S. Provisional Patent Application No. 63/400,479, filed Aug. 24, 2022, which is incorporated by reference herein in its entirety.

### FIELD OF THE INVENTION

Embodiments of the present invention relate generally to radiopaque materials and, in some cases, to biostable and/or biocompatible radiopaque components usable with implantable medical devices or medical instruments and tools.

### BACKGROUND OF THE INVENTION

Radiopaque materials are materials that are opaque when exposed to a x-ray or similar radiation. Often radiopaque materials are used as markers to indicate the location of something (e.g., an implant, a graft, or other implanted device). However, such radiopaque markers are hard substances and are not flexible, stretchable, or conformable-making their use and effectiveness in communicating information (e.g., through an x-ray) limited.

### BRIEF SUMMARY OF THE INVENTION

Embodiments of the present invention are directed towards radiopaque components for in vivo use with implantable devices (e.g., grafts, stents, catheters, leads, and other implants), tool used in surgeries (sponges), or surgical or medical procedural instruments (catheters components). The radiopaque component may be a flexible, stretchable, elastic, and/or conformable material that can be integral to or used with (e.g., attached to, positioned next to, etc.) the implantable device. The radiopaque component may be configured as a ribbon, a sheath, a rod, a string, a thread, a suture, a tube, a dot, an arrow, or other shape. The radiopaque component may be flat and/or two-dimensional or may be three-dimensional. In some embodiments, radiopaque component may be a coating for an implantable device or surgical tool or instrument, or a portion thereof. The radiopaque component may comprise a powdered or compounded radiopaque material entrapped within, encased within, encapsulated by, dispersed within, and/or intermixed within a binder. The binder may be biostable and/or biocompatible. In some embodiments, the radiopaque component may comprise at least 50% radiopaque material by weight, although other weight percentages are contemplated.

In some embodiments, the difference in specific gravity between the binder and the radiopaque material leads to more desirable distribution and encapsulation of the radiopaque material within the radiopaque component and enables the radiopaque material therein to be conformed to a desirable shape for use, such as with an implantable medical device. This uniformity and shapability, particularly while being encapsulated in a biostable and/or biocompatible binder, provides for improved x-ray readings and customizable options that have been unavailable until this invention.

In some embodiments, the radiopaque material (e.g., particles) may be dispersed or suspended within the binder after mixing. In other embodiments, this occurs in one step. In an embodiment, the radiopaque material comprises a plurality of particles and the plurality of particles are each fully coated, to the greatest degree possible, by the binder during the mixing step. After mixing, the binder is formed and cured and the radiopaque material may remain evenly distributed within the binder. The binder may provide flexibility, elasticity, stretchability, and/or conformability to the radiopaque component. In some embodiments, the radiopaque component may be flexible and/or bendable, and, in some embodiments, may stretch in one or more directions and return to its original form.

Depending on the desired end use, the radiopaque component may be formed into any shape, such as a rod, a dot, a ribbon, or other shape. Notably, different manufacturing techniques can be used to form the radiopaque components, such as injection molding, pultrusion, extrusion without molding (e.g., die extruding, optionally with a hydraulic ram), casting, vacuum molding, 3D printing, laser cutting, die cutting, amongst others. In this regard, the radiopaque component may be formed into complex shapes, such as with turns, curves, stepped heights, thinner portions, etc., and/or formed into shapes that impart specific information by viewing through an x-ray (such as an arrow, serial number, descriptor, etc.).

Some embodiments of the present invention use tantalum as the radiopaque material. Tantalum may be formed into particles (e.g., a fine powder) such that the weight of the tantalum particles does not cause the tantalum to settle within the binder prior to curing. Tantalum displays a similar radiopacity to that of gold and is less expensive to obtain; therefore using tantalum as the radiopaque component may be cost effective. Notably, however, other radiopaque materials can be used and formed into particles or compounds that are utilized in various embodiments of the present invention described herein. For example, gold or bismuth could be utilized in various embodiments of the invention.

Some embodiments of the present invention use a biostable and/or biocompatible binder, such as a silicone. The binder may be a curable silicone. The use of a curable silicone binder may induce a strong and enduring encapsulation of the radiopaque material within the silicone binder. In an embodiment, the silicone binder does not comprise any solvents that evaporate. Evaporation could cause undesirable alterations to the shape, size, thickness, or elastic characteristics of the radiopaque component. As solvents or other liquids evaporate from a mixture, the initial shape of the component must necessarily change. In an embodiment of the present invention, however, the inventors have overcome this issue by using a curable silicone binder which retains 100% of its starting volume, shape, size, thickness, and/or the like during curing. As such, the radiopaque materials maintain their position within the component during curing and later usage. Not only is it important that the radiopaque materials are maintained completely and securely within the binder, but it is also important that the radiopaque materials are maintained in their initial position. The inventors have accomplished that with the present invention. In some embodiments, the silicone may comprise room temperature vulcanizing silicone (RTV silicone). RTV silicone cures at room temperature and, as part of the invention, can comprise one-component or two-component silicone.

In some embodiments, an adhesive and/or overcoat may be used to attach (e.g., adhere, bind, etc.) the radiopaque component (i.e. the cured radiopaque material and binder) to the implantable device. For example, a silicone adhesive or other adhesive may be used. In an embodiment, the silicone adhesive comprises solvents that evaporate to a large degree, resulting in a significant reduction in volume (e.g., up to 50%, up to 70%, or up to 75% reduction in volume). The silicone components in the binder and adhesive may crosslink to one another, thereby forming a strong bond, while maintaining flexibility. In some embodiments, a compatible polymer, such as polyurethane, may be used to adhere the radiopaque component to the implantable device or medical tool or instrument. In some embodiments, other application methods are contemplated for attaching the radiopaque component, such as spraying an overcoating, applying the adhesive over the radiopaque component, mixing the radiopaque component with the adhesive and applying before curing, etc. Any application method known in the art may be utilized.

### BRIEF DESCRIPTION OF THE DRAWINGS

Having thus described the invention in general terms, reference will now be made to the accompanying drawings, which are not necessarily drawn to scale, and wherein:

FIG. 1A illustrates an example radiopaque component, in accordance with some embodiments discussed herein;

FIG. 1B illustrates the example radiopaque component shown in FIG. 1A adhered to example implantable medical devices, in accordance with some embodiment discussed herein;

FIG. 2A illustrates another example radiopaque component, in accordance with some embodiments discussed herein;

FIG. 2B illustrates the example radiopaque component shown in FIG. 2A adhered to example implantable medical devices, in accordance with some embodiment discussed herein;

FIG. 3A illustrates another example radiopaque component, in accordance with some embodiments discussed herein;

FIG. 3B illustrates the example radiopaque component shown in FIG. 3A attached to an example implantable medical device, in accordance with some embodiment discussed herein;

FIG. 4A illustrates another example radiopaque component, in accordance with some embodiments discussed herein;

FIG. 4B illustrates the example radiopaque component shown in FIG. 4A used to suture an example implantable medical device, in accordance with some embodiment discussed herein;

FIG. 5 illustrates a flow chart of an example method of forming a radiopaque component, in accordance with some embodiments discussed herein;

FIG. 6 illustrates a flow chart of another example method of forming a radiopaque component, in accordance with some embodiments discussed herein;

FIG. 7 illustrates a flow chart of another example method of forming a radiopaque component, in accordance with some embodiments discussed herein;

FIG. 8A illustrates two example aluminum steps, and a lead dot for use in calibration of an x-ray device, in accordance with some embodiments discussed herein;

FIG. 8B illustrates an example x-ray image corresponding to the calibration tools shown in FIG. 8A, in accordance with some embodiments of the present invention;

FIG. 9A shows an example calibration graph corresponding to the first aluminum step shown in FIG. 8A, in accordance with some embodiments discussed herein;

FIG. 9B shows an example calibration graph corresponding to the second aluminum step shown in FIG. 8A, in accordance with some embodiments discussed herein;

FIG. 10A illustrates an image of various radiopaque components configured for use with implantable medical devices, in accordance with some embodiments discussed herein;

FIG. 10B illustrates an x-ray image taken of the radiopaque components shown in FIG. 10A, in accordance with some embodiments discussed herein;

FIG. 11A illustrates an image of various radiopaque components configured for use with implantable medical devices, in accordance with some embodiments discussed herein;

FIG. 11B illustrates an x-ray image taken of the radiopaque components shown in FIG. 11A, in accordance with some embodiments discussed herein;

FIG. 12A illustrates an image of various radiopaque components configured for use with implantable medical devices, in accordance with some embodiments discussed herein;

FIG. 12B illustrates an x-ray image taken of the radiopaque components shown in FIG. 12A, in accordance with some embodiments discussed herein;

FIG. 13A illustrates an image of various radiopaque components configured for use with implantable medical devices, in accordance with some embodiments discussed herein;

FIG. 13B illustrates an x-ray image taken of the radiopaque components shown in FIG. 13A, in accordance with some embodiments discussed herein;

FIG. 14A illustrates an image of various radiopaque components configured for use with implantable medical devices, in accordance with some embodiments discussed herein;

FIG. 14B illustrates an x-ray image taken of the radiopaque components shown in FIG. 14A, at a first aluminum body thickness, in accordance with some embodiments discussed herein;

FIG. 14C illustrates an x-ray image taken of the radiopaque components shown in FIG. 14A, at a second aluminum body thickness, in accordance with some embodiments discussed herein;

FIG. 14D shows a graph depicting the variation in the equivalent aluminum thickness of the radiopaque components shown in FIGS. 14B-C, in accordance with some embodiments discussed herein;

FIG. 15A illustrates an image of various radiopaque components configured for use with implantable medical devices, in accordance with some embodiments discussed herein;

FIG. 15B illustrates an x-ray image taken of the radiopaque components shown in FIG. 15A, at a first aluminum body thickness, in accordance with some embodiments discussed herein;

FIG. 15C illustrates an x-ray image taken of the radiopaque components shown in FIG. 15A, at a second aluminum body thickness, in accordance with some embodiments discussed herein;

FIG. 15D shows a graph depicting the variation in the equivalent aluminum thickness of the radiopaque components shown in FIGS. 15B-C, in accordance with some embodiments discussed herein;

FIG. 16A illustrates an image of various radiopaque components configured for use with implantable medical devices, in accordance with some embodiments discussed herein;

FIG. 16B illustrates an x-ray image taken of the radiopaque components shown in FIG. 16A, at a first aluminum body thickness, in accordance with some embodiments discussed herein;

FIG. 16C illustrates an x-ray image taken of the radiopaque components shown in FIG. 16A, at a second aluminum body thickness, in accordance with some embodiments discussed herein;

FIG. 16D shows a graph depicting the variation in the equivalent aluminum thickness of the radiopaque components shown in FIGS. 16B-C, in accordance with some embodiments discussed herein;

FIG. 17A illustrates an image of various radiopaque components configured for use with implantable medical devices, in accordance with some embodiments discussed herein;

FIG. 17B illustrates an x-ray image taken of the radiopaque components shown in FIG. 17A, at a first aluminum body thickness, in accordance with some embodiments discussed herein;

FIG. 17C illustrates an x-ray image taken of the radiopaque components shown in FIG. 17A, at a second aluminum body thickness, in accordance with some embodiments discussed herein;

FIG. 17D shows a graph depicting the variation in the equivalent aluminum thickness of the radiopaque components shown in FIGS. 17B-C, in accordance with some embodiments discussed herein;

FIG. 18A illustrates two radiopaque components positioned about an implantable medical device, in accordance with some embodiments discussed herein;

FIG. 18B illustrates a computerized tomography (CT) scan image depicting the implantable medical device, including the two radiopaque components thereon, shown in FIG. 18A, implanted into a body, in accordance with some embodiments discussed herein;

FIG. 18C illustrates a three-dimensional CT scan image depicting a three-dimensional rendering of placement of the implantable medical device, including the two radiopaque components, shown in FIG. 18A, on the organ of the body, shown in FIG. 18B, in accordance with some embodiments discussed herein;

FIG. 18D illustrates a fluoroscopy scan image depicting the implantable medical device, including the two radiopaque components thereon, shown in FIG. 18A, implanted into a body, in accordance with some embodiments discussed herein;

FIG. 18E illustrates a magnetic resonance imaging image of a body having the implantable medical device, including the two radiopaque components thereon, shown in FIG. 18A, in accordance with some embodiments discussed herein.

FIG. 19A illustrates a photograph of various radiopaque components configured for use with implantable medical devices, in accordance with some embodiments discussed herein;

FIG. 19B illustrates an x-ray image of the radiopaque components shown in FIG. 19A;

FIG. 20A illustrates a photograph of various radiopaque components configured for use with implantable medical devices, in accordance with some embodiments discussed herein;

FIG. 20B illustrates an x-ray image of the radiopaque components shown in FIG. 19A;

FIG. 21A illustrates a photograph of various radiopaque components configured for use with implantable medical devices, in accordance with some embodiments discussed herein;

FIG. 21B illustrates an x-ray image of the radiopaque components shown in FIG. 19A;

FIG. 22A illustrates a photograph of various radiopaque components configured for use with implantable medical devices, in accordance with some embodiments discussed herein;

FIG. 22B illustrates an x-ray image of the radiopaque components shown in FIG. 19A;

FIG. 23A illustrates a photograph of various radiopaque components configured for use with implantable medical devices, in accordance with some embodiments discussed herein;

FIG. 23B illustrates an x-ray image of the radiopaque components shown in FIG. 19A;

FIG. 24 illustrates an example calibration curve for equivalent aluminum thickness;

FIG. 25 illustrates a diagram of an example method of making a radiopaque component of the invention;

FIG. 26 illustrates a diagram of an alternative method of making a radiopaque component of the invention;

FIG. 27 illustrates a diagram of another alternative method of making a radiopaque component of the invention;

FIG. 27 illustrates a photograph illustrating a radiopaque component woven into a fabric;

FIG. 29A illustrates a photograph of various radiopaque components configured for use with implantable medical devices, in accordance with some embodiments discussed herein;

FIG. 29B illustrates an x-ray image of the radiopaque components shown in FIG. 29A;

FIG. 29C illustrates an x-ray image of the radiopaque components shown in FIG. 29A; and

FIG. 30 illustrates an example calibration curve for equivalent aluminum thickness.

### DETAILED DESCRIPTION OF THE INVENTION

Some example embodiments now will be described more fully hereinafter with reference to the accompanying drawings, in which some, but not all example embodiments are shown. Indeed, the examples described and pictured herein should not be construed as being limiting as to the scope, applicability or configuration of the present disclosure. Rather, these example embodiments are provided so that this disclosure will satisfy applicable legal requirements. Like reference numerals refer to like elements throughout.

Radiopaque component(s) may be formed from a radiopaque material and a binder material. The radiopaque material and the binder material may be intermixed such that the binder material entraps the radiopaque material therein. In an embodiment, the entrapment or encapsulation of the radiopaque material within the binder is a physical or mechanical entrapment or encapsulation. That is, in an embodiment, the binder surrounds the radiopaque particles or material. In an embodiment, there is no chemical bonding (e.g. permanent chemical attachment such as via an ionic or covalent bond) which occurs between the radiopaque material and the binder. In an embodiment, the binder does not etch the surface of the radiopaque component. In an embodiment, the radiopaque component is solvent-free. In an embodiment, the radiopaque component comprises solely a binder and a radiopaque material.

In some embodiments, the radiopaque material may be tantalum (Ta), gold (Au), bismuth (Bi) and/or barium (Ba), or a combination thereof. Similarly in some embodiments, the radiopaque material may be a compound containing one or more of the radiopaque materials, for example bismuth chloride (BiCl₃), barium sulfate (BaSO₄), and tantalum oxide (Ta₂O₄). In some embodiments, the radiopaque material may be gold or tantalum, such as if minimized thickness of the radiopaque component is a priority, while, in other embodiments, a bismuth compound or BaSO₄ may be used, such as if a larger thickness may be desired. In some embodiments, barium or bismuth may be provided in another form that is biostable, biocompatible, or not bioreactive and could be utilized as the radiopaque material in such form.

In an embodiment, the radiopaque material may comprise a particular phase of tantalum—α, β, or a combination thereof. Tantalum has a stable a phase and a metastable β phase. Tantalum's α phase is amorphous and possesses good chemical, thermal and mechanical properties, along with good ductility and formability, but is relatively ductile and soft. Tantalum's β phase is crystalline, more hard and brittle, and has a higher resistivity. It has a tetragonal structure, whereas the α phase has a body-centered cubic structure. β tantalum has been found to have a fiber texture and distinct orientation relative to the substrate, which is the same irrespective of the crystalline nature of the substrate. In an embodiment, the β tantalum may have a more active K-edge than α tantalum. In an embodiment, the radiopaque material comprises α tantalum and in another embodiment, the radiopaque material comprises β tantalum. In an embodiment, the radiopaque material comprises an a tantalum core (e.g., a rod, as described herein) and β tantalum is utilized on the exterior of the radiopaque component. For example, in an embodiment, a yarn (e.g. a polymer yarn) which is braided about the α tantalum core (e.g., a suture, as described herein) may be sputter coated with β tantalum. In an embodiment, the radiopaque material comprises commercially pure tantalum powder.

The radiopaque material may comprise at least 50% by weight of the radiopaque component, although other percentages by weight are contemplated (and the desired percentage by weight may depend on the radiopaque material chosen and/or the binder(s) chosen). For example, as detailed herein, when using tantalum in a silicone binder at a certain thickness, 50%-95% by weight of the tantalum has shown desirable viewing results through an x-ray-showing equivalent opacity viewing to much thicker aluminum, for example. In some embodiments, the radiopaque material may comprise tantalum (in a silicone binder) at up to about 92% by weight of the radiopaque component. In some embodiments, the radiopaque material may comprise tantalum (in a silicone binder) at up to about 95% by weight of the radiopaque component.

In this regard, the radiopaque material prevents x-rays from penetrating through the radiopaque component, such that the radiopaque component is readily visible on an x-ray image. In some embodiments, the radiopaque material may be configured as particles (e.g., a powder, such as a finely divided powder) and/or compound. In some embodiments, the plurality of powder particles are small enough to remain suspended within a binder prior to being cured. Said differently, the plurality of powder particles may be sized such that the density of the powder does not cause the plurality of powder particles to settle within the binder. In some embodiments, the plurality of particles may be sized by and/or to a mesh so as to meet a desired particle size. In an example embodiment, the plurality of particles may be a 325 mesh, although other mesh sizes are contemplated. Notably, in some embodiments, another size customizable device may be used, while in some embodiments, a size customizable device may not be used.

Any size of the particles is contemplated and may depend on the binder(s) and/or radiopaque material type being used. For example, in some embodiments, each of the plurality of particles may be less than 90 microns in diameter, less than 50 microns in diameter, about or less than 44 microns in diameter, less than 30 microns in diameter, about or less than 25 microns in diameter, less than 10 microns in diameter, or even about or less than 1 micron in diameter. In some embodiments, each of the plurality of particles may be between 10 microns and 45 microns in diameter. In some embodiments, each of the plurality of particles may be between 10 microns and 90 microns in diameter. In some embodiments, each of the plurality of particles may be between 30 microns and 45 microns in diameter. In a particular embodiment, each of the plurality of particles may be about 25 microns±3 microns, in diameter. In some embodiments, each of the plurality of particles may be between 60-400 mesh. In some embodiments, each of the plurality of particles may be between 10-250 microns.

In an embodiment, the radiopaque particles may be tantalum and may be nodular, angular, and/or spherical in shape. Nodular tantalum powder may be made by molten sodium reduction of tantalum salt or solid magnesium reduction of tantalum oxide. The powder comprises aggregates consisting of primarily particles and pores. It has relatively low density and high surface area. Angular tantalum powder may be made by hydride and crush of a solid ingot (i.e. electron beam or vacuum arc melted), and de-hydride. The particle looks angular and is non-aggregated; its bulk density is typically higher than that of nodular.

In other embodiments, radiopaque particles may be spherical in shape. Spherical particles of tantalum, for example, can be formed from nodular or angular particles of tantalum via inert gas atomization techniques, plasma spheroidization, or a plasma rotating electrode process (PREP). Any process known in the art for producing spherical tantalum (or other similar particles) may be utilized herein. In an embodiment, the spherical particles of tantalum have an average aspect ratio between 1.0 and 1.1. In an embodiment, the spherical particles of tantalum, when disposed within a silicone carrier, may be in closer contact with one another and may, therefore, create a denser radiopaque material. Likewise, the spherical particles in close contact may reduce the volume of oxygen or other gases within the radiopaque material, additionally adding to the density of the material. In an embodiment, radiopaque components formed with spherical tantalum may be stronger than radiopaque components formed with tantalum of other shapes.

In an embodiment, spherical particles of tantalum may have an improved flow rate. In an embodiment, the Hall Flow rate for the particles used as the radiopaque component may be less than about 15 sec/50 g. In another embodiment, the Hall Flow rate for the particles used as the radiopaque component may be between 5 sec/25 g and 15 sec/25 g. In yet another embodiment, the Hall Flow rate for the particles used as the radiopaque component may be between 5 sec/25 g and 8 sec/25 g. In some embodiments, the particles may be up to 6N purity, oxygen levels may be low (e.g., approximately 100-400 ppm), the particles may be free of voids, and/or the particles may carry no satellites. In some embodiments, the particles may have a carbon, nitrogen, iron, nickel, and/or chromium content of less than 50 ppm. In an embodiment, the particles may have an apparent density of between about 5 and 10 g/cc. In another embodiment, the particles may have an apparent density of between about 8 and 10 g/cc. In yet another embodiment, the particles may have a tap density of between about 8 and 12 g/cm³.

In some embodiments, the binder material may be a biocompatible and/or biostable material. In this regard, the binder material may not be harmful to living tissue within the body and/or may be chemically stable when positioned within the body. In some embodiments, accordingly, the radiopaque component may form no byproducts when introduced into the body. The binder material may further impart flexibility, shapability, and/or conformability on the radiopaque component. In this regard, the radiopaque component may be configured to be shaped into a component which may be, for example, movable about an implantable device, or moldable about the implantable device. The binder material may also cause the radiopaque component to be smooth, in comparison to other devices. In this regard, the radiopaque component may be “soft” since the radiopaque component may be flexible and smooth, rather than abrasive. In some embodiments, the radiopaque component may be stretchable or have elastic properties such that the original shape of the radiopaque component may stretch to some degree (e.g., up to 2% its original length/size, up to 5%, up to 10%, up to 20%, up to 30%, up to 50%, etc.), which may aid in desired conformity, positioning, and/or attachment to an implantable medical device.

In some embodiments, the binder material may be a silicone medium. In some embodiments, the silicone medium is an FDA approved silicone for implantation. In some embodiments, the silicone may be a silicone elastomer. In some embodiments, the binder (e.g. silicone) may be linear. In some embodiments, the binder material may be a two-part silicone system that is mixed in use. In an embodiment, the binder material may be translucent. In an embodiment, the binder material may be thixotropic. In an embodiment, the binder material may have a high tear strength. In an embodiment, the binder does not comprise a reaction product of a diisocyanate, a polymeric aliphatic diol, or a chain extender. In an embodiment, the binder does not comprise an endgroup.

In some embodiments, the binder material may be configured to cure at room temperature, and, in some embodiments, heat may be applied (e.g., an oven, heat gun, lamp, or any other method known in the art) to decrease the cure time. In some embodiments, the binder material may be cured with low or no atmospheric moisture to cure, while in other embodiments, the binder material may require atmospheric moisture to cure. In some embodiments, the binder material may not produce any off-gas or byproducts during or after curing. In other embodiments, the binder material may produce byproducts or may off-gas during or after curing. In an embodiment, the silicone may have a specific gravity between 1.0 and 1.2.

The radiopaque material and the binder material may be intermixed, shaped and cured to form the radiopaque component. In some embodiments, the radiopaque component may be at least 50% radiopaque material by weight, at least 60% radiopaque material by weight, at least 80% radiopaque material by weight, at least 90% radiopaque material by weight, or at least 96% radiopaque material by weight, before curing. In some embodiments, the radiopaque component may be between 50%-96% radiopaque material by weight prior to curing.

The radiopaque material may define a larger density than the binder material. In this regard, although there is a larger weight percent of the radiopaque material, in comparison to the binder material, the binder material may define a larger volume percent than the radiopaque material. Thus, the radiopaque material may be intermixed (e.g., randomly distributed, evenly distributed, etc.) and entrapped within the binder material prior to curing to form the radiopaque component.

In some embodiments, the binder material may be configured to suspend the radiopaque material before and during the curing process. To explain, it may be desirable for the radiopaque material to be evenly distributed throughout the radiopaque component, such that the entire radiopaque component exhibits the same coloration and is easily visible on an x-ray image, even to an untrained eye. The binder may form a matrix about the radiopaque materials, maintaining the radiopaque materials in close proximity to each other, in an embodiment. It is also worth noting that, additionally, the radiopaque component is visible to the naked eye without an x-ray, such as may be beneficial to a surgeon while it is being implanted (e.g., to enable proper initial positioning in the body). Thus, the radiopaque material may be distributed throughout the binder solution while the binder material cures. In this regard, a binder material defining a greater uncured viscosity may be able to overcome the greater density of the radiopaque material and hold the radiopaque material in suspension. Said differently, the weight of each of the plurality of radiopaque particles is unable to overcome the viscosity of the binder material, and thus, remains suspended.

In some embodiments, to prevent settling the radiopaque material may be a finely divided powder. As discussed, since the radiopaque material defines a greater density than the binder material, any large clumps or particles of the radiopaque material may settle within the binder material due to the density difference. In contrast, in a finely divided powder each of the plurality of powder particles comprise minimal weight such that the radiopaque material powder remains suspended within the binder material throughout the curing process.

In some embodiments, the uncured binder material may have a viscosity of at least 50,000 cP, at least 60,000 cP, at least 70,000 cP, or at least 80,000 cP. In an embodiment, the binder may be a silicone binder with a viscosity between about 70,000 and 90,000 cP. In this regard, upon mixing the uncured binder material with the radiopaque material, the binder material may entrap and suspend the radiopaque material. Said differently, the binder material and the radiopaque material may be intermixed. In some embodiments, the binder material completely surrounds each of the radiopaque material particles.

In this regard, the difference in specific gravity between the binder and the radiopaque material leads to more desirable distribution and encapsulation of the radiopaque material within the radiopaque component and enables the radiopaque material therein to be conformed to a desirable shape for use, such as with an implantable medical device. This uniformity and shapability, particularly while being encapsulated in a biostable and biocompatible binder, provides for beneficial x-ray readings.

Prior to or in conjunction with curing, the radiopaque mixture may be formed into different shapes. In some embodiments, the radiopaque mixture may be molded, formed, and/or combined with other materials to form radiopaque components for application with an implantable medical device or may be formed as an integral part of an implantable medical device or medical tool. Notably, different manufacturing techniques can be used to form the radiopaque components, such as injection molding, die cutting, amongst others. In this regard, the radiopaque component may be formed into complex shapes, such as with turns, curves, stepped heights, thinner portions, etc., and/or formed into shapes that impart specific information by viewing through an x-ray (such as an arrow, serial number, descriptor, etc.). Curing may be accomplished simultaneous with the forming process in some embodiments.

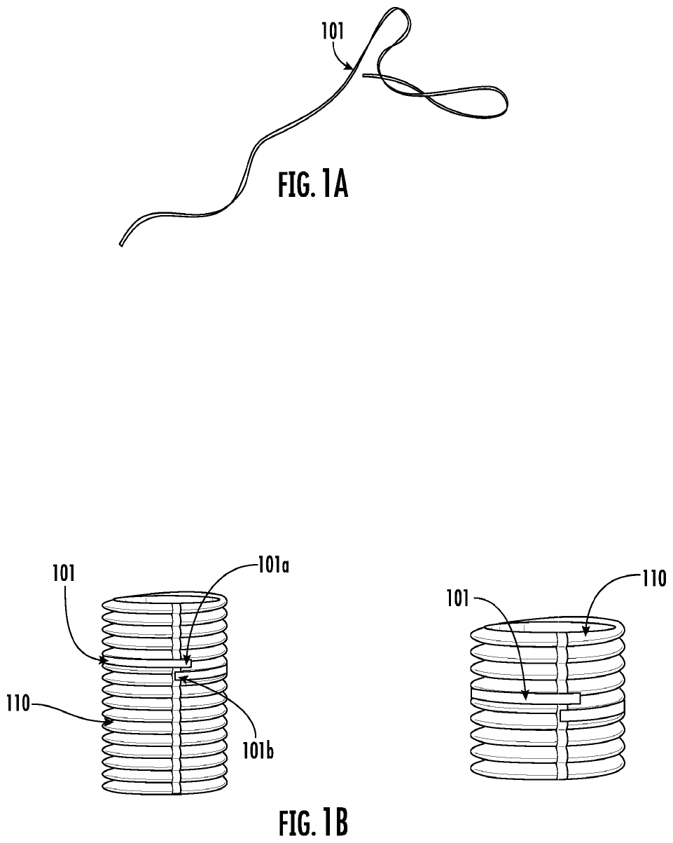

FIGS. 1A-4B illustrate example radiopaque components and exemplary uses on implantable devices. In some embodiments, illustrated in FIG. 1A the radiopaque component may be configured as a rod 101. Use of the term “rod” herein shall not imply that the radiopaque component is necessarily rigid. In fact, in some embodiments, the radiopaque rod may be extensible, conformable, flexible, and/or elastic. The term “rod” may, in some embodiments, reflect a thread, strand, fiber, or the like. The term “rod” may, but need not necessarily, comprise a cylindrical shape. In an embodiment, a radiopaque rod of the invention may be cut into discrete lengths. Such cutting may be accomplished via a knife, blade, or laser.

In some embodiments, the rod 101 radiopaque component may comprise at least 80% radiopaque material by weight, at least 85% radiopaque material by weight, at least 90% radiopaque material by weight or even at least 95% radiopaque material by weight of the radiopaque component, prior to curing. In some embodiments, the greater the weight percent of radiopaque material by weight, the more “matte” or the less translucent the appearance of the radiopaque component.

In some embodiments, the rod 101 radiopaque component may be flexible and may exhibit two-way stretch. In this regard, the rod 101 may be able to stretch lengthwise. In some embodiments, the rod 101 may be able to stretch up to 20%, up to 25% or even up to 30%, where the stretch is measured by the change in length of the rod 101 from a relaxed position to an extended position under a given tension or load. In some embodiments, the stretch may be even greater. For example, the inventors found that the stretch of a 10 cm (length) 0.5 mm (diameter) rod comprising 90% spherical tantalum may be as high as 165%. Further, the rod 101 may be flexible. In this regard, the rod 101 may be configured to bend, twist, braid, fold, and/or compress without adverse effect.

As is understood in the art, a Shore durometer is a device for measuring the hardness of a material, such as a polymer. Higher numbers on the scale indicate a greater resistance and are thus harder materials. Lower numbers indicate less resistance and are thus software materials. In an embodiment, the radiopaque component may have a Shore hardness A (or a Shore durometer) of between about 15 and 95, about 20 and 70, or about 50. The selection of a binder (as discussed herein) and/or methodology used may affect the Shore durometer of the radiopaque component. For example, a binder could itself have a starting Shore A hardness of 20, as an example, contributing to the overall Shore A hardness of the radiopaque component.

In some embodiments, the rod 101 may be positioned around an implantable medical device 110, illustrated in FIG. 1B. In some embodiments, the implantable medical device 110 may be an artificial artery, a graft, or other device which may be three-dimensional in shape. Thus, the rod 101 may be adhered about a perimeter of a cross-section of the implantable medical device 110. The rod 101 may be adhered about a portion of the implantable medical device 110 in an embodiment.

In some embodiments, in addition to providing a location of the implantable medical device 110, the rod may be used to indicate the diameter, length, width, size, shape, or orientation of the implantable medical device 110 when presented on an x-ray image. This may provide a surgeon or other doctor with information about the implantable medical device 110, for example if there has been any change (e.g., enlarging, shrinking, twisting, movement, etc.) of the implantable medical device. In some embodiments, the rods 101, for example where a first end 101a and a second end 101b are positioned, may be used to inform if the implantable medical device is twisting within the body. For example, if the first end 101a and the second end 101b were positioned on the top of the implantable medical device when implanted, and at a follow up procedure were located on a side of the implantable medical device it would indicate that the implantable medical device is moving within the patient. Similarly, the general shape of the rod may be oblong, spiral, or twisted on the x-ray-thereby indicating twisting of the graft. In some embodiments, the rod may be positioned along the length of the graft, which also may be used to determine if twisting of the graft has occurred.

In some embodiments, the rod 101 may be used as a guide for an ultrasound or similar device for additional imaging or follow up. In some embodiments, rod(s) 101 may be used to indicate the location of branches on grafts. For example, a rod 101 may be positioned on the main artery, and then a second rod may be positioned about the branched artery adjacent the main artery. In other embodiments, the rod 101 may be formed into a spiral shape around a graft or other medical device or may fully or partially circumvent or form a ring around a graft or other medical device.

Notably, while such example benefits are described with respect to the rod shape, one of ordinary skill in the art will readily appreciate that similar benefits can be obtained by other shapes, some such other shapes being described herein.

In an embodiment, while the shape is described as a rod, it should be understood that the radiopaque component may be described differently-such as a string or thread or fiber. In some embodiments, the rod 101 (or other shape) may be less than 1 millimeter in diameter or thickness, less than 0.75 millimeters in diameter or thickness, less than 0.5 millimeters, or less than 0.2 millimeters in diameter or thickness. In this regard, the rod 101 may be configured to nest within ribbing or on the exterior of the implantable medical device 110.

In some embodiments, the rod 101 may be adhered to the implantable medical device 110 with an adhesive. In some embodiments, the adhesive may be a silicone adhesive, particularly if the binder is a silicone binder. In some embodiments, the silicone adhesive be disposed between the radiopaque component and the medical device and/or may also cover the radiopaque component, adding to the biostability and biocompatibility thereof. In some embodiments, the silicone in the adhesive and the silicone in the radiopaque rod 101 may crosslink to form a bond. Similarly, in some embodiments, the implantable medical device 110 may at least partially comprise silicone, thus, the silicone adhesive may form crosslinking bonds between the radiopaque rod 101 and the implantable medical device 110.

In some embodiments, the rod 101 may be molded. In this regard, the mold may transfer serial numbers or other imprints into the rod. Thus, the rod, in addition to providing an indication of relative position (or other information, such as twist, etc.), the rod, through the ex-ray, may provide additional readable information. Notably, any other shapes, including for example the dot or ribbon illustrated herein, may be formed to include additional readable information, such as a serial number or other imprint. In some embodiments, the mold may comprise fluorinated ethylene propylene (FEP).

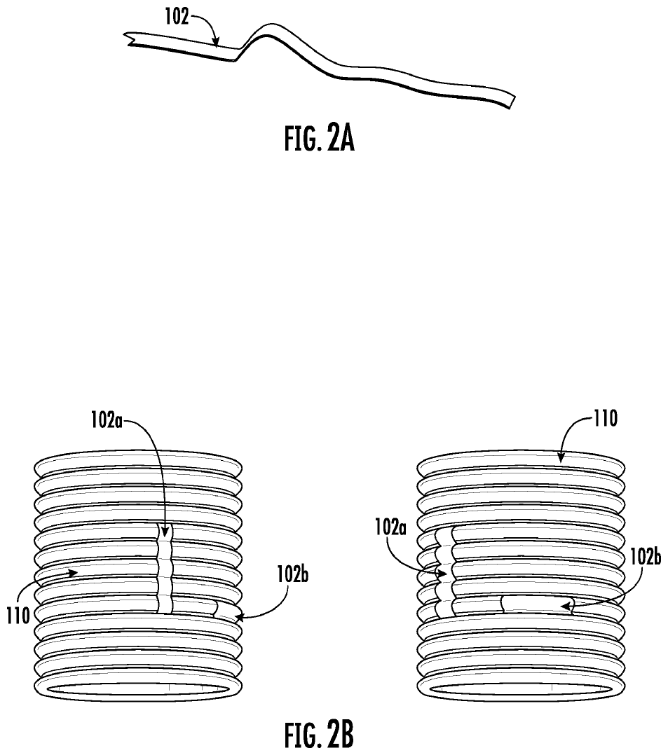

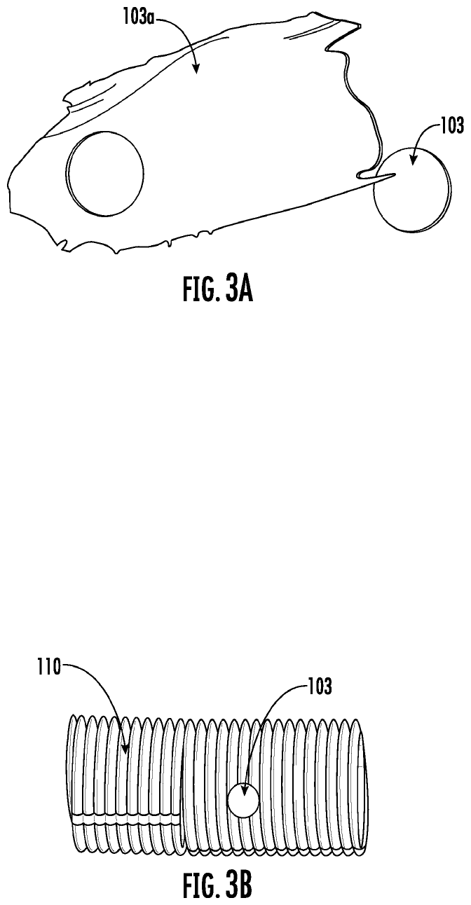

In some embodiments, illustrated in FIGS. 2A-B, the radiopaque component may be configured as a strip or ribbon 102 (e.g., a flattened rod). In some embodiments, the ribbon 102 may define a width between 0.1-2.5 mm, between 0.15-2.3 mm, or even between 0.2-2.0 mm. In some embodiments, the ribbon 102 may define a thickness of at least 0.05 mm, at least 0.1 mm or at least 0.15 mm thick. In some embodiments, the ribbon 102 may define a thickness of about 0.05 mm or less, about 0.1 mm or less or about 0.15 mm or less. In some embodiments, a ribbon 102 defining a larger thickness may be easier to locate on an x-ray image taken from any direction. To explain, if the thickness of the ribbon 102 is aligned with the x-ray, the ribbon 102 on the resulting image may not be as readily visible, as if the narrow side of the ribbon 102 was aligned with the x-ray.

In some embodiments, illustrated in FIG. 2B, the ribbon 102 may be adhered to the implantable medical device 110. The ribbon 102 may be adhered with a silicone adhesive, such that the adhesive creates crosslinking bonds between the ribbon 102 and the implantable medical device 110 as described with relation to the rod (e.g., 101 FIG. 1B).

In some embodiments, the ribbon 102 may be configured to conform to the surface of the implantable medical device. In this regard, if the implantable medical device defines a ribbed surface, the ribbon 102 may be configured to contour to the surface of the implantable medical device such that the length of the ribbon contacts the surface of the implantable medical device.

In some embodiments, the ends of the rod 101 and/or ribbon 102 may be configured to indicate a direction relative to the implantable medical device. For example, in an embodiment, an end of a ribbon or rod may be cut or formed into an arrow shape, such as to indicate a useful direction. As noted above, the ribbon or rod may be formed to indicate other information, such as readable information (e.g., a serial number or other imprint).

In some embodiments, a first ribbon 102a and a second ribbon 102b may be positioned on the implantable medical device 110 perpendicular to one another. This configuration may allow a better position reading on an x-ray image as it is likely that one of the either the first ribbon 102a or the second ribbon 102b will be in the plane of the x-rays. For example, in image 1 the first ribbon 102a extends within the plane of the x-ray while, the second ribbon 102b is more angled. Thus, in the resulting x-ray image the first ribbon 102a would be easily identifiable, while the second ribbon 102b may be harder to identify. In contrast, in image 2 the first ribbon 102a is angled with respect to the x-ray, while the second ribbon 102b is aligned with the plane of the x-ray. Thus, in image 2, the second ribbon 102b may be easier to identify, in comparison to the first ribbon 102a.

In some embodiments, the radiopaque component may be configured in a planar shape 103 as illustrated in FIG. 3A. In some embodiments, the planar shape 103 may be a circle, a square, a donut-shape, or similar closed shape. In some embodiments, the planar shape 103 may comprise a larger weight percent of the radiopaque powder. The planar shape 103 may be, in an embodiment, punched out of a sheet 103a of the radiopaque component. In some embodiments, the sheet 103a is a clay-like consistency prior to curing. In this regard, the sheet 103a may be shaped into a desired configuration, rather than pushing the material into a mold, for formation. The sheet 103a, prior to curing, may be cut into the desired planar shape 103 for use with the implantable medical device 110 as illustrated in FIG. 3B.

In some embodiments, the planar shape 103 may be customizable. For example, in some embodiments, the planar shape may define a diameter of at least 1 mm, at least 2 mm, or at least 3 mm. In some embodiments, the planar shape 103 may define a diameter of up to 9 mm, up to 6 mm, or up to 3 mm. In some embodiments, the planar shape 103 may define a thickness of less than 1.5 mm, less than 1.3 mm, or less than 1.0 mm. In some embodiments, the planar shape may define a thickness of at least 0.1 mm.

In some embodiments, such as illustrated in FIG. 3B, the planar shape 103 may be adhered onto the implantable medical device 110. In this regard, a silicone adhesive may be used as discussed with reference to FIGS. 1A-B. In some embodiments, the planar shape 103 may be sewn on to the implantable medical device 110. In some embodiments, the planar shape 103 may be sewn onto the implantable medical device 110 with a suture or other suitable means.

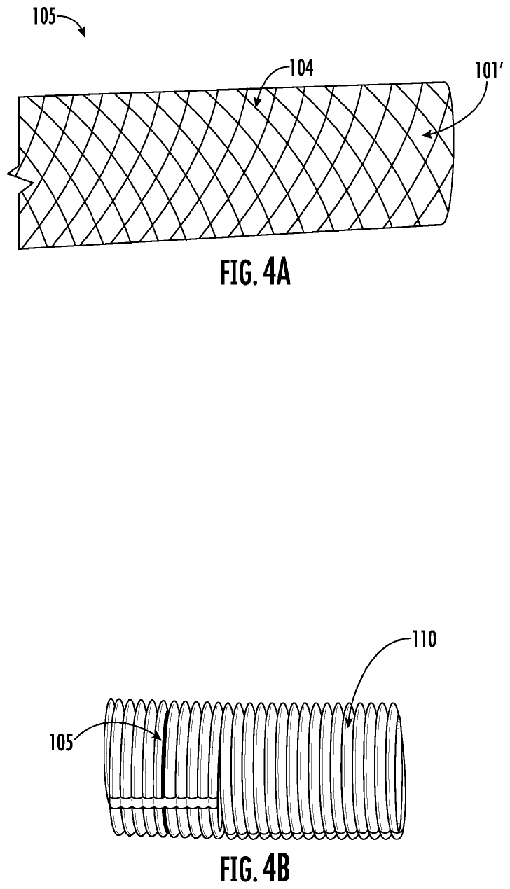

In some embodiments, such as illustrated in FIGS. 4A-B, the radiopaque component may be configured as a suture or thread 105. In use as a thread, the thread can be woven into fabric, polymeric, or any other woven materials (see FIG. 28). In this embodiment, the radiopaque thread can be woven in weft and/or warp directions and/or sewn in as a suture. The inventive weaving technique may be useful to provide a marker in a woven graft, for example. In this embodiment, the use of a weft and a warp radiopaque marker may allow a visual inspection of the marker (via x-ray) to determine whether a tubular graft is open or closed. This can be an important indicator of undesirable graft infolding complications.

In some embodiments, the suture 105 may comprise a rod 101′ of the radiopaque component (e.g., FIG. 1A) and a wrapped, encasing, or braided material 104 (e.g. a polymer) surrounding the rod 101′. In an embodiment, the radiopaque component may comprise one or more yarns of the braided material 104—as an alternative to the rod 101′ being a radiopaque component or in addition thereto. Thus, the radiopaque component may be braided about a core (e.g. a polymeric core) to form the suture. In some embodiments, the braided material 104 may be configured to provided structural integrity to the rod 101′.

In some suture embodiments, radiopaque material 104 may be extruded about a core material. In an embodiment, the core material may comprise a polymer. Any polymeric material known in the art may be utilized as the core material. In some embodiments, the polymeric core material may comprise polyester, polypropylene, polyethylene, polyethylene terephthalate, nylon or any other polymer. In some embodiments, the polymer may have an inherent degree of elasticity. In other embodiments, the polymer may not be elastic or extensible. In a particular embodiment, the core material may comprise ultra high molecular weight (UHMW) polyethylene. The polymeric core may comprise a denier of, in an embodiment, less than 1, less than 3, less than 5, less than 7, less than 10, or between 10 and 400. In some embodiments, the polymeric core may comprise a denier of between about 3 and 40. In some embodiments, the polymeric core may comprise a denier of between about 5 and 20.

In an embodiment, the polymeric core may be flexible in a transverse direction, but may have a low or very low degree of stretch or elongation in a longitudinal direction. In other embodiments, the polymeric core may have a reasonable degree of stretch or elongation in the longitudinal direction. In any of the embodiments discussed herein, an additive may be utilized within the polymeric core and/or the radiopaque material (discussed below) which provides elasticity to the final product (e.g. spandex, elastane).

In an embodiment, the radiopaque material may be extruded around the polymeric core such that the polymeric core is encapsulated within the radiopaque material. The polymeric core may be centered within the radiopaque material in some embodiments, but may not be in other embodiments, radiopaque material may be extruded around the polymeric core such that the polymeric core is encapsulated within the radiopaque material. In some embodiments, the length of the radiopaque suture increases during stretching and the diameter of the radiopaque suture decreases. In this embodiment, the polymeric core may not stretch. In some embodiments, the radiopaque material may adhere to the polymeric core. In other embodiments, a primer (i.e. silane) may be added to increase adhesion sites between the radiopaque material and the polymeric core. After curing, in an embodiment, the inventive suture has a low degree of stretch in the longitudinal direction (i.e. 0% to 1% extensibility), but remains flexible.

In another embodiment, the inventive suture may comprise a radiopaque material core rather than a polymeric core. The radiopaque core may comprise a denier of, in an embodiment, less than 1, less than 3, less than 5, less than 7, less than 10, or between 10 and 400. In some embodiments, the radiopaque core may comprise a denier of between about 3 and 40. In some embodiments, the radiopaque core may comprise a denier of between about 5 and 20. In this embodiment, one or more thin polymeric fibers (again, using any polymer known in the art, including but not limited to UHMW polyethylene) may be braided about a blunt cannula (or any other similar device) to form a cylindrical, helically wound braid. The tension may then be released on the cylindrical braid by removing the cannula. Uncured radiopaque material of the invention may then be injected into the longitudinal cavity of the braided cylindrical polymer. As the radiopaque materials extends through the cylindrical polymer braid, the natural forces of the braid with released tension stretch the radiopaque material. After curing, the final form of the suture (the braided polymer about a radiopaque core) may have little to no stretch or extensibility in the longitudinal direction.

In some embodiments, the rod 101′ used within the suture 105 may comprise a lower radiopaque material by weight in comparison to the rod 101 to be adhered to the implantable medical device 110. However, in other embodiments, the rod 101′ used within the suture 105 may comprise a similar amount of radiopaque material by weight as the rod 101 designed to be adhered to an implantable medical device 110. Notably, the suture 105 may be visible on an x-ray image to indicate the presence of the suture. As such, the suture 105 can be used in connection with any medical device or procedure. For example, the suture 105 could be used to close an internal organ in a procedure that does not involve any implantable medical devices at all. The suture 105 could be x-rayed to determine whether the organ incision has reopened or remains closed and intact.

In an embodiment, the suture 105 may be a 3-0 suture, 4-0 suture, 5-0 suture, 6-0 suture, 7-0 suture, 8-0 suture, 9-0 suture, or the like. In an embodiment, the rod 101′ used in the suture is the equivalent of a 9-0 suture or an 8-0 suture and when the wrapped or braided 104 material encases the rod 101′, the suture is the equivalent of a 5-0 suture or 6-0 suture. In an embodiment, the sutures of the invention meet the U.S. Pharmacopeia requirements for sutures (http://www.pharmacopcia.cn/v29240/usp29nf24s0_m80190.html).

In an embodiment, the radiopaque particles or component within the suture 105 may be segmented or intermittent, such that certain sections of the suture 105 contain the radiopaque particles or component and other sections of the suture 105 do not contain the radiopaque particles or component. In an embodiment, the radiopaque component is disposed throughout the suture 105, but the radiopaque particles within the radiopaque component are intermittent or segmented within the radiopaque component.

In some embodiments, the rod 101′ may comprise up to 50% radiopaque material by weight. In other embodiments, the rod 101′ may comprise greater than 50% radiopaque material by weight—for example, 90% radiopaque material by weight. In either case, the rod 101′ may retain flexibility and stretchability imparted by the binding material. In some embodiments, the braided material 104 may be a flexible, semi-rigid, rigid, biostable, and/or biocompatible material. In some embodiments, the braided material 104 may comprise polyethylene terephthalate (PET), although other materials are contemplated (e.g., expanded polytetrafluoroethylene (ePTFE), polytetrafluorethylene (PTFE), polypropylene, ultra-high molecular weight polyethylene (UHMWPE), polyethylene, nylon, etc.). In some embodiments, such as illustrated in FIG. 4B, the thread 105 may be used to attach the radiopaque compound to medical devices (e.g., grafts), such as at an opening 110, and/or close an opening formed in the implantable medical device.

Although as discussed herein the radiopaque component is used with an implantable medical device such that the implantable device may be easily identified in an x-ray, the radiopaque component may be attached to other devices. For example, the radiopaque component may be molded to surgical instruments, threaded into sponges, and other tools that are used in surgery, such as to be certain that none of the devices are left in the patient. In some embodiments, the radiopaque component may be integrally layered into a medical device, instrument, or tool. For example, a medical device, instrument or tool may comprise a plurality of layers and the radiopaque component may be one of the layers thereof. Likewise, the radiopaque component may be printed, painted, coated, or sprayed onto a medical device, instrument or tool, such as to provide a radiopaque coating. The coating may coat the entirety of the medical device, instrument or tool or may coat only a portion thereof, such as printed, painted, coated, or sprayed in a line, arrow, or “T” shape on the medical device, instrument or tool. Any shape or design is contemplated herein.

In an embodiment, the radiopaque component may be encased in a hard shell or rigid casing to protect it. For example, in connection with a percutaneous dilating catheter tip for the deployment of a stent, the radiopaque component may be encased in a rigid polymeric shell and affixed to the distal end of a catheter to be used in or as a catheter tip. The rigidity of the shell may allow movement and placement of the catheter. Similarly, the radiopaque component can be over-molded onto an existing catheter tip or other medical instrument or device. Other rigid encasements are contemplated herein, including but not limited to use with medical instruments and tools that are used in surgical procedures. The hard encasing shell could be any polymeric material discussed herein, such as a thermoplastic polyurethane.

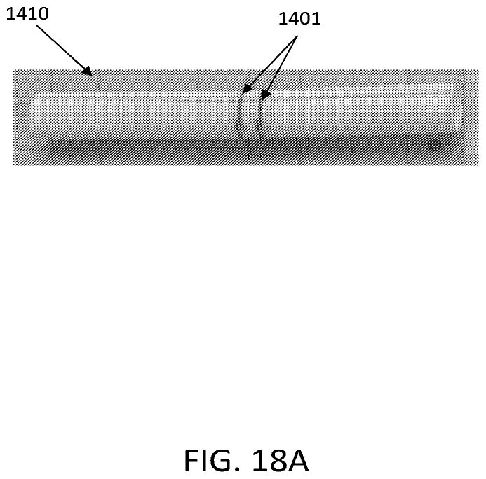

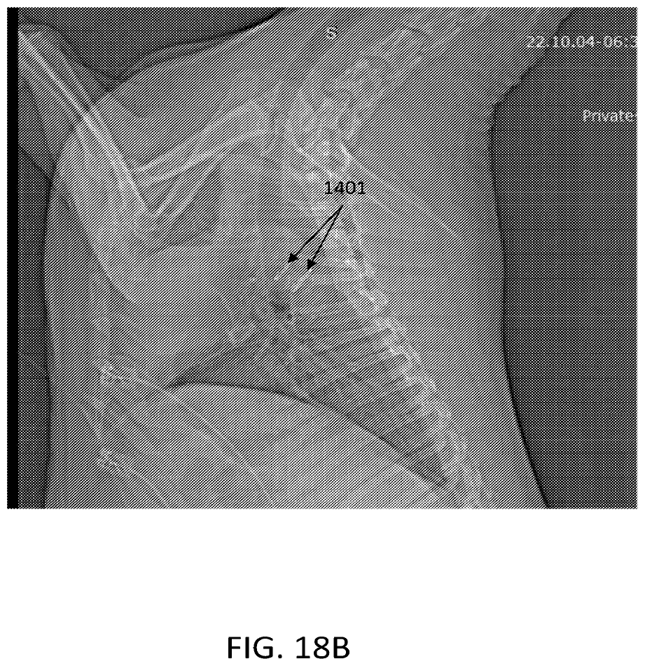

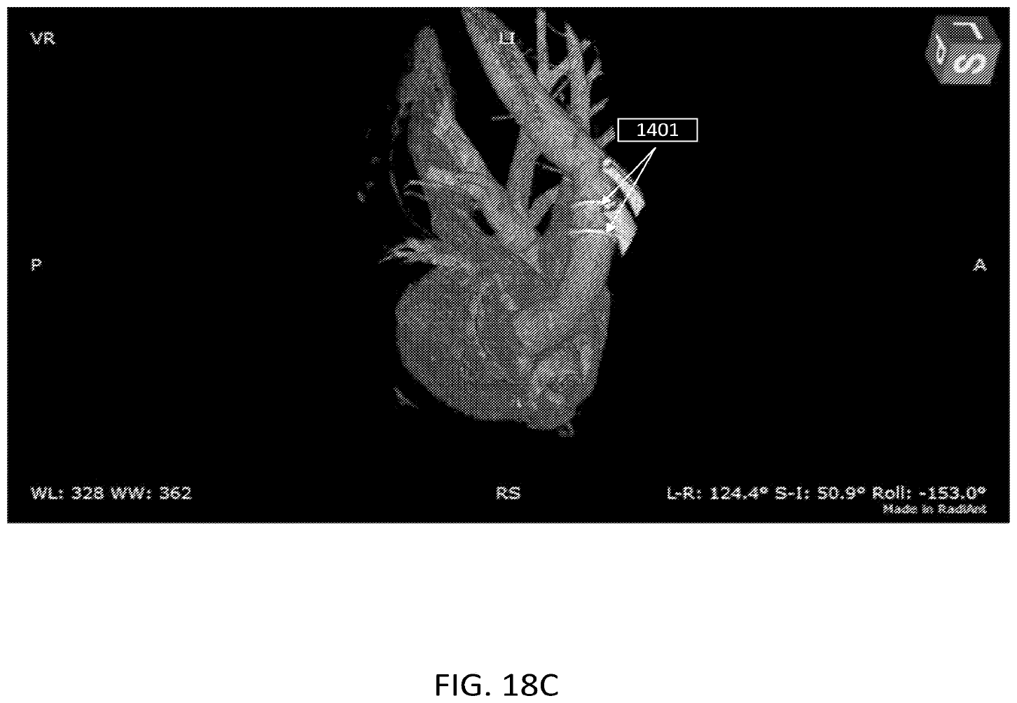

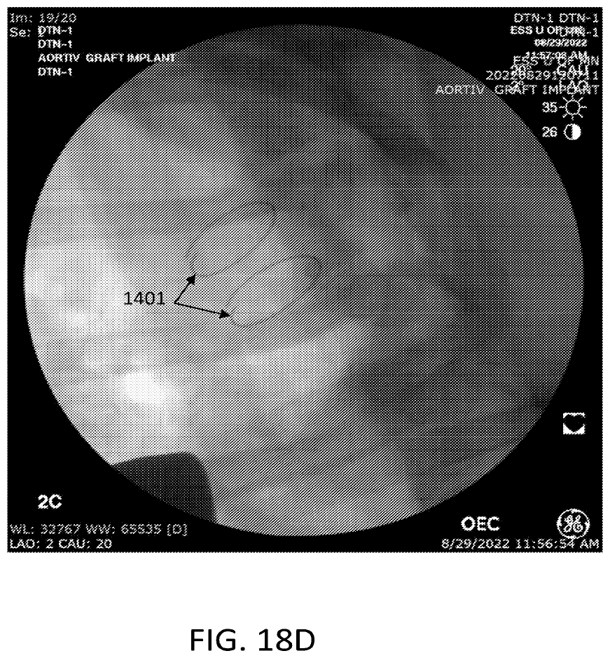

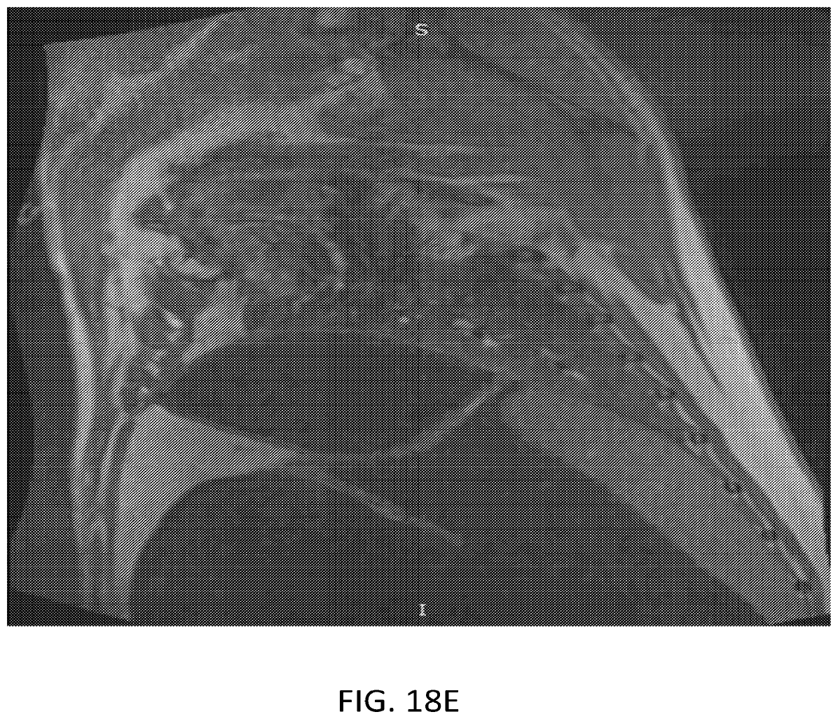

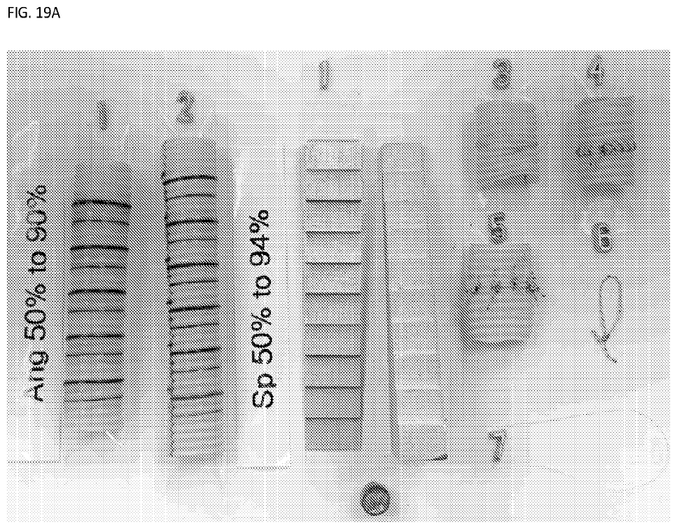

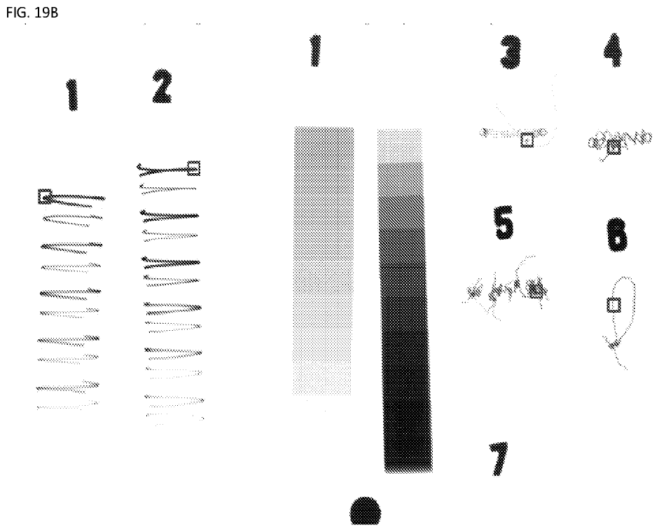

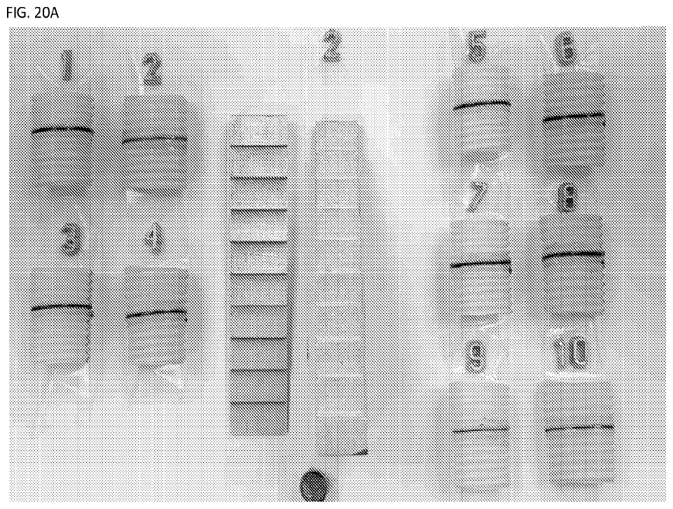

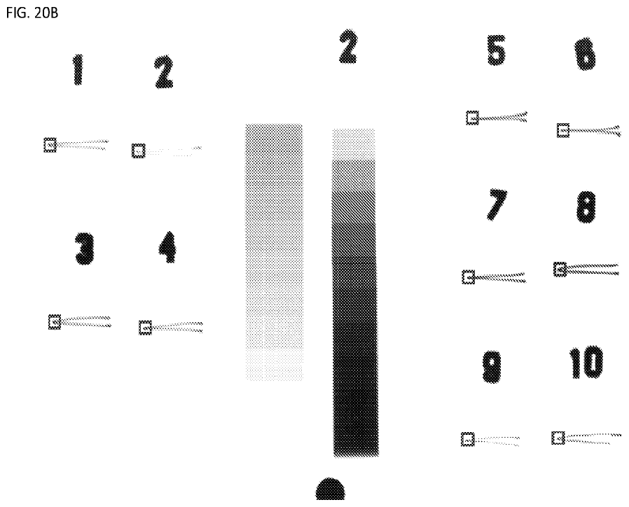

Scatter radiation is a type of secondary radiation that occurs when the beam intercepts an object, causing the radiation to be scattered. Scatter creates noise and distracts from a clear image. In an embodiment, the radiopaque component causes minimal or no scatter in magnetic resonance imaging (see e.g., FIG. 18E). and does not interfere with imaging. In an embodiment, the radiopaque material is not damaged or negatively affected by magnetic resonance imaging. In an embodiment, the implanted device is not damaged or negatively affected by the affixed radiopaque component or material during magnetic resonance imaging.

### Example Flowchart(s) and Operations

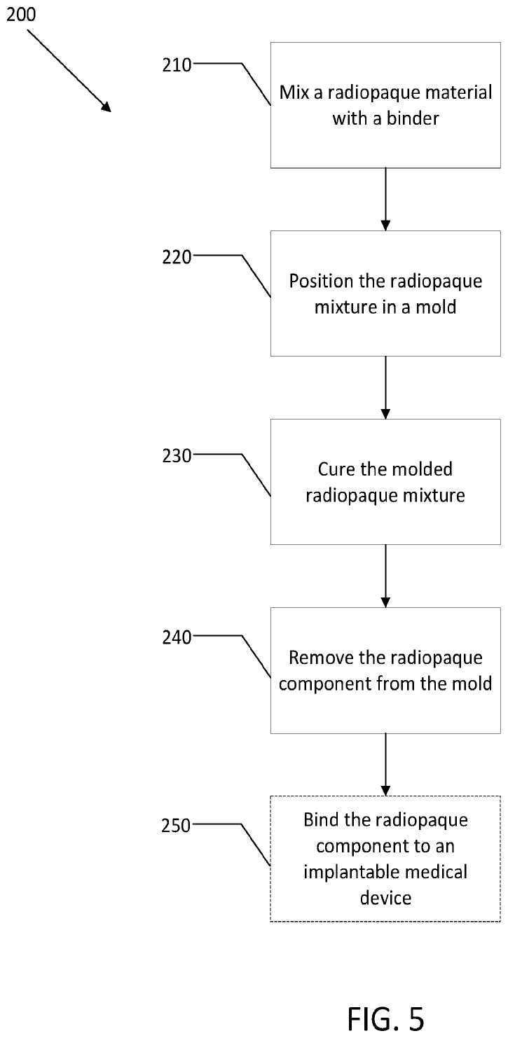

Some embodiments of the present invention provide methods and apparatus related to forming the radiopaque components according to various embodiments described herein. Various examples of the operations performed in accordance with embodiments of the present invention will now be provided with reference to FIGS. 5-7.

FIG. 5 illustrates a flow chart according to an example method 200 of creating a radiopaque component. At operation 210, a radiopaque powder is mixed with a binder material. In some embodiments, the radiopaque powder may be a tantalum powder. In some embodiments, the binder material may comprise one or more of a silicone, a silicone mixture, a thermoplastic polyurethane, an aliphatic polycarbonate, an aliphatic polycarbonate-based thermoplastic polyurethane, a thermoplastic silicone polyurethane co-polymer, a polycarbonate, and/or one or more thermoplastic elastomers, or combinations thereof. In some embodiments, the radiopaque material may be at least 80% by weight of the total mixture. In some embodiments, the radiopaque material and the binder material may be mixed in an oxygen-free or low-oxygen environment, such as within a low-oxygen or oxygen-free bag. Excluding or limiting oxygen may additionally prevent or reduce bubbles from forming within the mixture, thereby preventing voids within the injected mixture and final molded composition. In some embodiments, the radiopaque material and the binder material may be mixed in a humidity and/or moisture-free or low moisture and/or low humidity environment (e.g., a nitrogen box). Excluding or limiting moisture or humidity may extend the working time of the uncured mixture, allowing more time for the injection and/or molding process to occur. In some embodiments, the mixture may be squeegeed or compressed in all directions until the radiopaque material is intermixed with the binder material, such that each particle of the radiopaque material is entrapped in the binder material.

In an embodiment, the binder and radiopaque materials are intermixed without use of a solvent, reducing the time, expense and complexity of the process. In this embodiment, the binder is not dissolved in a solvent prior to mixing with the radiopaque material and no evaporation process is required to remove any liquid solvent from the mixture.

At operation 220, the radiopaque mixture is positioned into a mold. In some embodiments, the radiopaque mixture may be retrieved by a syringe, and injected into the respective mold (e.g., injection molding). In some embodiments, the mold may be tubular, such as to make the rod (e.g., 101 FIG. 1A), while in other embodiments, the mold may be flat, such as to make the ribbon (e.g., FIG. 2A). In some embodiments, a vacuum may be positioned on one end of the mold so as to overcome the viscosity of the mixture, to pull the entire radiopaque mixture into the mold (e.g., vacuum molding). In other embodiments, the radiopaque mixture may be extruded (see FIG. 25).

In an embodiment, after extruding the radiopaque mixture, injecting the radiopaque mixture, or otherwise molding the radiopaque mixture into, for example, a rod, sheet, or ribbon, the radiopaque mixture is stretched. Any method of stretching known in the art is contemplated herein, such as a manual stretching technique (e.g., molding the rod within a FEP mold which can be uniformly axially stretched) or drawing the mixture through a set of rollers operating at different speeds, the second set of rollers operating at a faster speed than the first set of rollers. The stretching may occur under heat or at room temperature. In an embodiment, the rod or ribbon is stretched in the machine direction (longitudinally, MDO) or in the transverse direction (latitudinally, TDO). In other embodiments, the ribbon or a sheet, for example, may be biaxially stretched (stretched in the machine direction and transverse direction). In an embodiment, the radiopaque mixture is stretched to a thickness or diameter, as the case may be, which is up to half of its original thickness/diameter. In an embodiment, the radiopaque mixture is stretched to a thickness or diameter, as the case may be, which is up to 75% of its original thickness/diameter. In an embodiment, the radiopaque mixture is stretched to a thickness or diameter, as the case may be, which is up to 25% of its original thickness/diameter. In some embodiments, the stretching may occur while the radiopaque mixture is within the mold. While not wishing to be bound by theory, it is believed that the stretching process may contribute to the improved radiopacity of the radiopaque component.

In an embodiment, the stretching may occur via a pultrusion technique. For example. FIGS. 25 and 26 illustrate diagrams of a pultrusion method which may be utilized with or without extrusion. A molded (FIG. 25) radiopaque component may be disposed continuously on a spool 201 for the pultrusion process. Alternatively, the process may comprise extrusion (FIG. 26) via an extruder 300, directly into pultrusion. Any variation on these techniques known in the art is contemplated herein. In an embodiment, the radiopaque component is rolled about at least one roller 202, 301, 302 designed to hold the radiopaque component in place. In an embodiment, the radiopaque component then passes through a heating device 203, 303, such as a tube. In an embodiment, the radiopaque component is stretched via tension applied from a pull mechanism 204, 304. The pull mechanism 204, 304 may comprise two squeeze rollers which turn in opposite directions (clockwise/counterclockwise) to pull the radiopaque material therethrough. In an embodiment, the squeeze roller 204, 304 may spin/rotate at a higher rate than that of the roller 202, 301, 302, thereby stretching the radiopaque material positioned between the roller 202, 302 and the squeeze rollers 204, 304, under heat. In an embodiment, the stretched radiopaque rod may then continue to a cutting device 205, 305. The heating device 203, 303 may aid in curing the radiopaque material.

At operation 230, the radiopaque mixture is cured. In some embodiments, the radiopaque mixture may be cured at room temperature, while in other embodiments the radiopaque mixture may be heat cured. In some embodiments, the radiopaque mixture may be catalyst activated. In some embodiments, the curing time is dependent on the temperature of the surroundings. In some embodiments, the curing time may be up to 12 hours, up to 24 hours, or up to 72 hours. In a particular embodiment, the radiopaque mixture may be cured at 100° C. for about 15 minutes. In an embodiment, the curing process of the radiopaque mixture is a chemical process in which the silicone mixture undergoes a chemical reaction to change from gel or liquid to solid, to be contrasted from evaporation (loss of liquid components from the mixture). In this embodiment, the radiopaque mixture and/or radiopaque component are chemically cured. In an embodiment, there are no liquid components within the silicone mixture to evaporate.

At operation 240, the radiopaque component is removed from the mold. In some embodiments, the radiopaque component may be removed manually from the mold, while in other embodiments the radiopaque component may be removed hydraulically from the mold with alcohol. In some embodiments, additional cutting, shaping or sizing may be completed after removal from the mold. For example, the radiopaque component (e.g., 101 FIG. 1A) may be trimmed to a desired length, or a directional component may be added to the radiopaque component (e.g., an arrow on a ribbon 102 FIG. 2A). After the radiopaque component is removed from the mold, it may exhibit elastic, flexible and/or stretchable properties.

Optionally, at operation 250, the radiopaque component may be attached to an implantable medical device. In some embodiments, a silicone adhesive may be used to adhere the radiopaque component to the implantable medical device. In some embodiments, the silicone adhesive may be biostable and/or biocompatible, and may increase the biostability and/or the biocompatibility of the radiopaque component. In some embodiments, the silicone adhesive may be applied only between the radiopaque component and the implantable medical device, while in other embodiments the adhesive may also be applied over the radiopaque component as a coating.

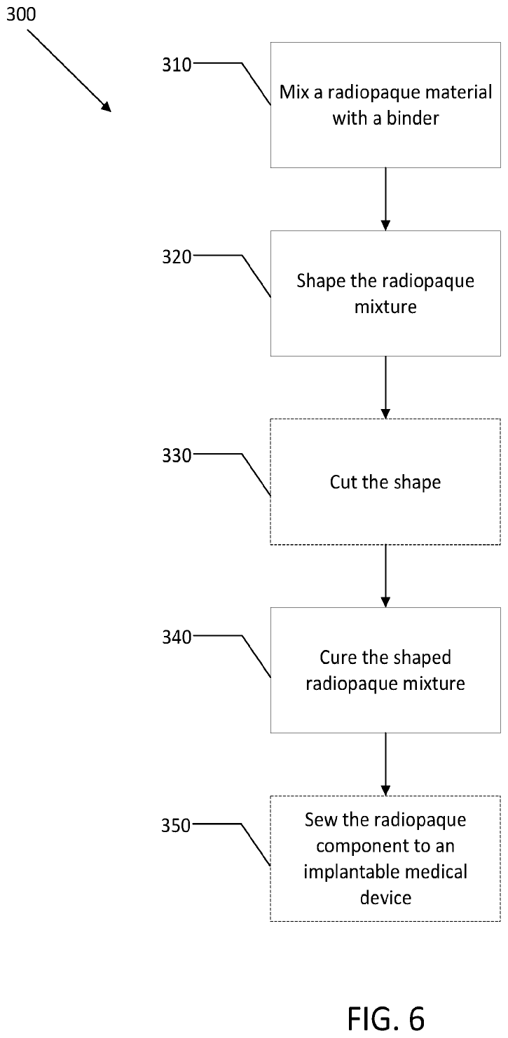

FIG. 6 illustrates a flow chart according to an example method 300 of creating a radiopaque component. At operation 310, a radiopaque material is mixed with a binder material. In some embodiments, the radiopaque material may be a radiopaque powder, for example, tantalum powder. In some embodiments, the binder material may be a silicone mixture. In some embodiments, the radiopaque material may be more than 90% by weight of the total mixture. In some embodiments, the radiopaque material and the binder material may be mixed in an airless environment, such as between two airless layers of plastic. In some embodiments, the mixture may be squeegeed in all directions until the radiopaque material is intermixed with the binder material, such that each particle of the radiopaque material is entrapped in the binder material.

At operation 320, the radiopaque mixture is shaped. In some embodiments, the radiopaque mixture is rolled into a thin sheet (e.g., 103a FIG. 3A), while in other embodiments, the radiopaque mixture is formed into a planar shape (e.g., 103 FIG. 3A). Notably, a thin sheet shape may be useful in various medical applications, such as in valve leaflets.

Optionally, at operation 330, the shaped radiopaque mixture is cut into one or more planar shapes (e.g., 103 FIG. 3A). In some embodiments, multiple planar shapes may be cut from the shaped radiopaque mixture.

At operation 340, the planar shapes may be cured. As discussed with relation to FIG. 5, the radiopaque component may be cured in a variety of ways—at room temperature, in an oven (e.g., exposed to heat) or due to a catalyst, as examples. In some embodiments, the cure time may be up to or at least 12 hours, at least 24 hours, or at least 16 hours.

Optionally, at operation 350, the radiopaque component may be attached to an implantable medical device. In some embodiments, the radiopaque component may be sewn onto the implantable device, while in other embodiments a silicone (or other) adhesive may be used.

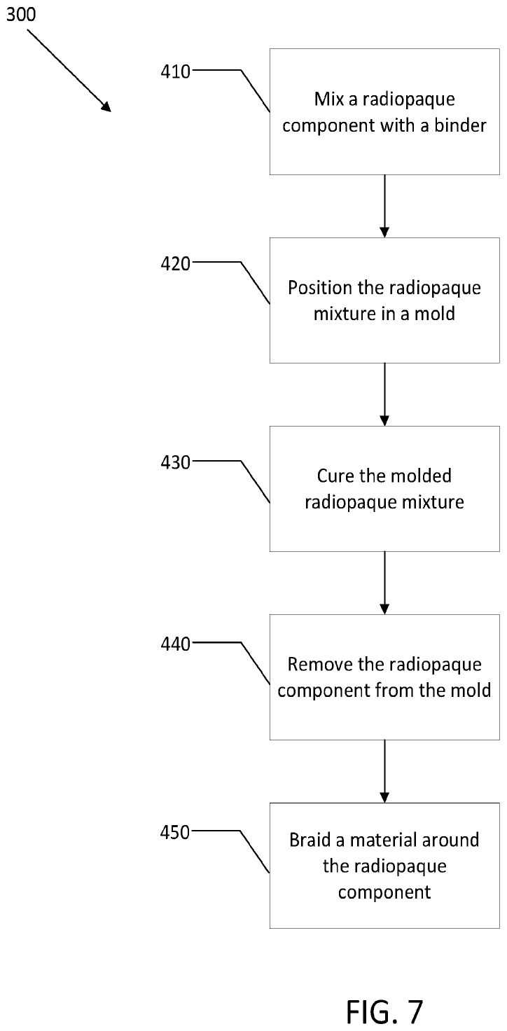

FIG. 7 illustrates a flow chart according to an example method 400 of creating a radiopaque thread component. At operation 410, a radiopaque material is mixed with a binder material. In some embodiments, the radiopaque material may be a radiopaque powder, for example tantalum powder. In some embodiments, the binder material may be a silicone mixture. In some embodiments, the radiopaque material may be about 50% by weight of the total mixture. In some embodiments, the radiopaque material and the binder material may be mixed in an airless environment, such as between two airless layers of plastic, within a vacuum chamber, within a nitrogen chamber, etc. In some embodiments, the mixture may be squeegeed in all directions until the radiopaque material is intermixed with the binder material, such that each particle of the radiopaque material is entrapped in the binder material.

In some embodiments, the radiopaque mixture may be retrieved by a syringe, and injected into the respective mold. In some embodiments, the mold may be tubular, such as to make the rod (e.g., 101 FIG. 1A). In some embodiments, a vacuum may be positioned on one end of the mold so as to overcome the viscosity of the mixture, to pull the entire radiopaque mixture into the mold.

At operation 430, the radiopaque mixture within the mold is cured. In some embodiments, the radiopaque mixture may be cured at room temperature, while in other embodiments the radiopaque mixture may be heat cured. In some embodiments, the radiopaque mixture may be catalyst activated. In some embodiments, the curing time is dependent on the temperature of the surroundings. In some embodiments, the curing time may be up to 12 hours, up to 24 hours, or up to 72 hours.

At operation 440, the radiopaque component is removed from the mold. In some embodiments, the radiopaque component may be removed hydraulically with alcohol or another solvent.

At operation 450, a material may be braided around the radiopaque component. In some embodiments, the material may be PET, expanded polytetrafluoroethylene (ePTFE), polytetrafluorethylene (PTFE), polypropylene, ultra-high molecular weight polyethylene (UHMWPE), polyethylene, nylon, or a similar structural material configured to impart structural integrity on to the radiopaque component such that the braided radiopaque component may be used as a thread in sutures and other stitches, interlaces, or loops.

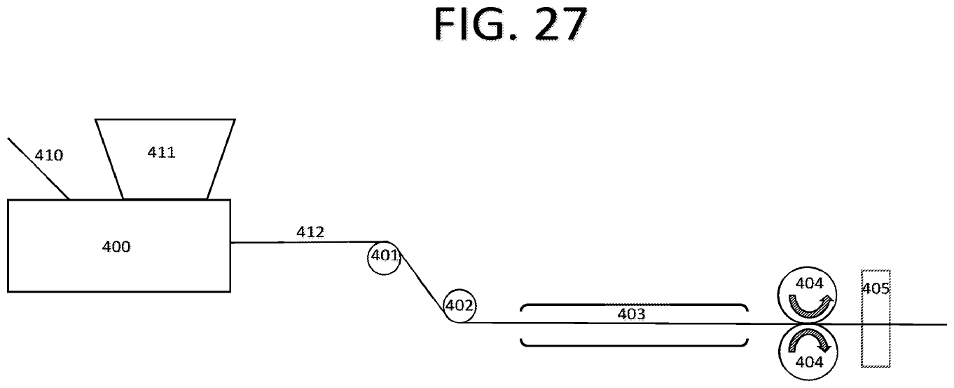

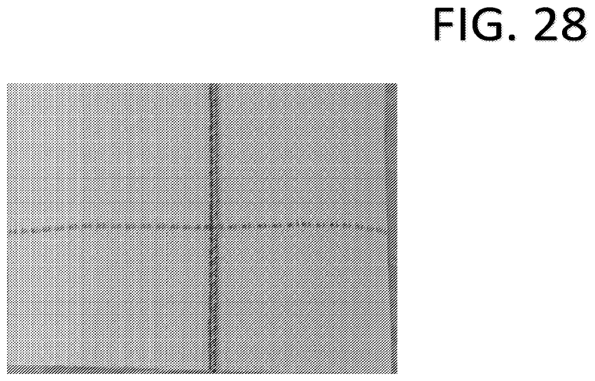

FIG. 27 illustrates a diagram wherein a radiopaque component which comprises a polymeric core and a radiopaque sheath is formed. In this embodiment, a polymeric yarn 410 (e.g. polyethylene terephthalate, ultra high molecular weight polyethylene, a resorbable polymer, or any other polymer) may be fed into an extruder 400. This polymeric yarn 410 may be fed into the extruder via a loom shuttle (not pictured) or any other mechanism known in the art. In an embodiment, the tension of the pultrusion mechanism set forth in FIG. 27 is controlled by the loom shuttle pad spring. In an embodiment, an uncured liquid radiopaque mixture 411 is also fed into the extruder 400. The uncured liquid radiopaque mixture 411 may be fed into the extruder 400 by a syringe pump (not pictured) or any other mechanism known in the art. In an embodiment, the thickness of the radiopaque component 412 may be controlled by the syringe pump. The uncured liquid radiopaque mixture 411 is disposed about the polymeric yarn 410 and the combined uncured liquid radiopaque mixture 411 and polymeric yarn 410 is extruded to form a radiopaque component 412 comprising a radiopaque sheath or coating about the polymeric yarn. In an embodiment, the radiopaque component 412 is then optionally stretched and cured as discussed above. The overall speed of the extrusion and/or pultrusion process may be controlled with a stepper motor and a microprocessor. A separate microprocessor may be used to control the temperature in the heating device 403.

### EXAMPLES

### Example 1

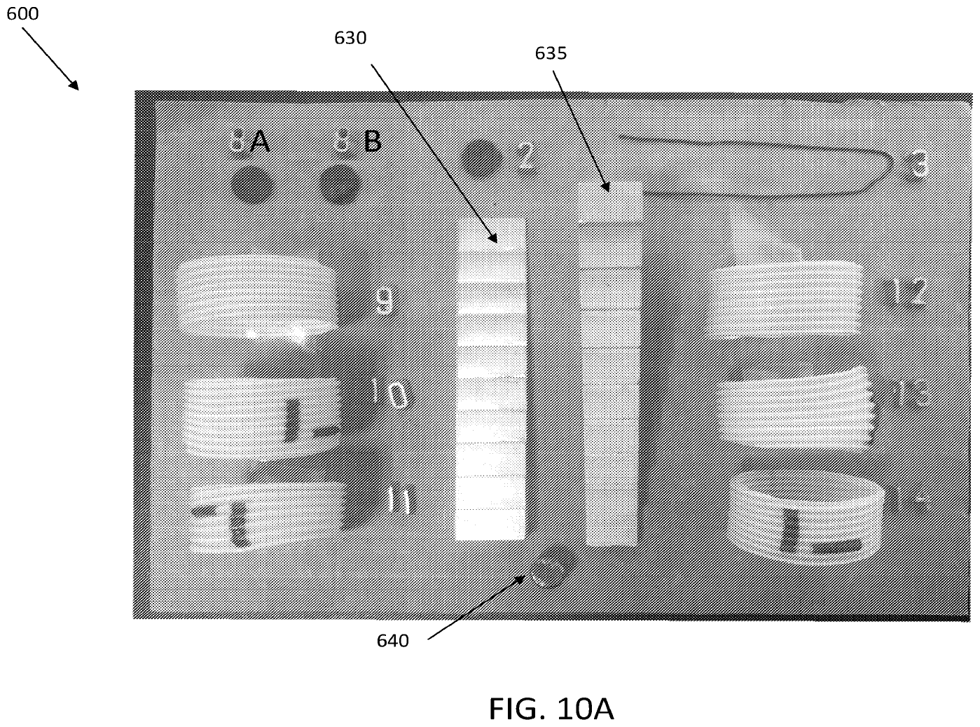

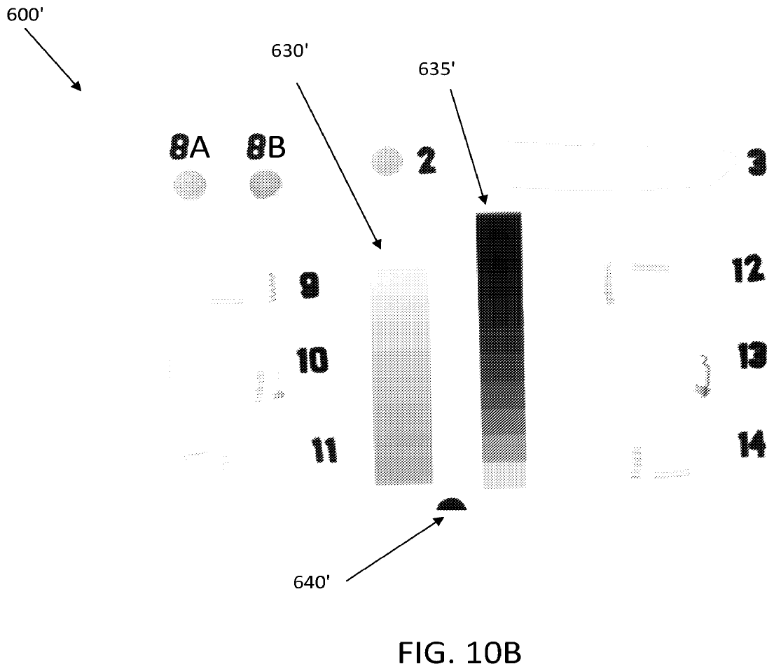

In an experiment, the radiopacity of different radiopaque components (e.g., 101, 102, 103, 105 FIGS. 1A, 2A, 3A, 4A) comprising different amounts of radiopaque powder was determined and was compared to known radiopaque components. The x-ray radiopacity was determined per ASTM F640-20 using a digital image and processing. Various digital images were taken (e.g., FIG. 10A) to show the positioning and orientation of the radiopaque components either on an implantable device, or directly on a backing paper. Such digital images were taken prior to or after an x-ray image such that a corresponding x-ray image thereof (e.g., FIG. 10B) may be easier to interpret. For example, in radiopaque components which may not be as identifiable, the reader of the image may be guided to a position to see if there is contrast in the x-ray image about that point.

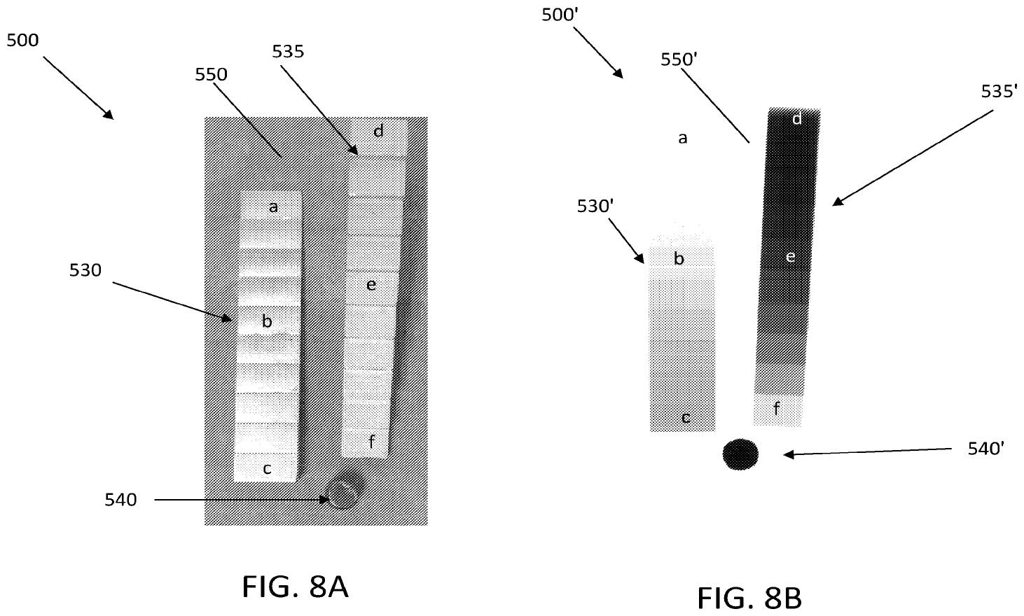

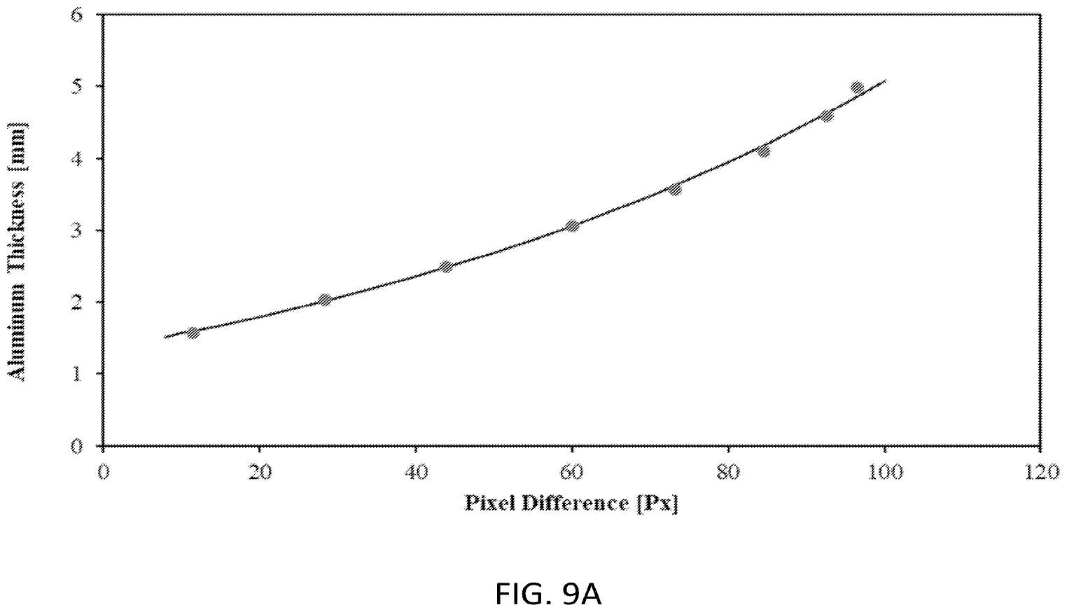

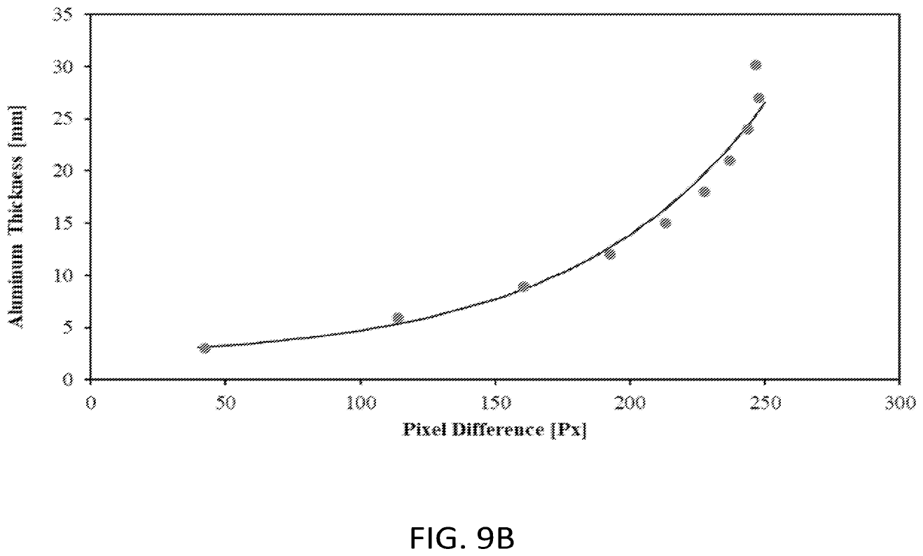

In each trial, the system is calibrated to calculate the radiopacity as compared to an equivalent aluminum thickness. A calibration method is illustrated in FIGS. 8A-9B. FIG. 8A shows a digital image 500 of a calibration setup. The calibration setup includes a first aluminum wedge 530, a second aluminum wedge 535, and a lead dot 540 positioned on a backing paper 550. The first aluminum wedge 530 comprised 10 steps, each about 0.5 mm high with the shortest step (a) being about 0.5 mm tall, and the tallest step (c) being 5.0 mm tall. The second aluminum wedge 535 comprised 10 steps, each about 3 mm high with the shortest step (f) being about 3 mm and the tallest step (d) being about 30 mm tall.

The calibration set up was x-ray imaged to obtain an x-ray image 500′ of the calibration set up, shown in FIG. 8B. To obtain an x-ray image, x-rays are directed at an object. The x-rays may be absorbed or scattered by the radiopaque component such that the x-rays are unable to reach the detector film or plate. Thus, the radiopaque component is visible under normal x-ray conditions. Using the x-ray image 500′, the pixel intensity was measured at different parts of the image including the background 550′, each step of the first aluminum wedge 530′, each step of the second aluminum wedge 535′ and the lead dot 540′. In analyzing the x-ray image 500′ for the pixel intensity of each step in the first aluminum wedge 530′ and the second aluminum wedge 535′ and the lead dot 540′, a pixel measurement was taken from the portion of the image 550′ being analyzed, and three control background measurements from the portions of the background 550′ closest to the portion of the image 550′ were measured. The multiple background measurements were to ensure consistency across the image 550′, as there may be variability across the background 550′ of some images 500′ and thus using a single baseline background image may not be accurate for calculating the effective thickness.

The difference in the grayscale pixel density between the sample and the background was calculated to provide a measure of radiopacity. The calibration results for each of the first wedge 530 and the second wedge 535 are provided in Tables 1 and 2, respectively.

TABLE 1

| Thickness | Wedge | Background | Pixel |
|---|---|---|---|
| [mm] | [Px] | Contrast [Px] | Difference [Px] |
| 1.57 | 243.5 | 255 | 11.5 |
| 2.03 | 226.6 | 255 | 28.4 |
| 2.50 | 211.1 | 255 | 43.9 |
| 3.05 | 194.9 | 255 | 60.1 |
| 3.57 | 181.8 | 255 | 73.2 |
| 4.10 | 170.5 | 255 | 84.5 |
| 4.59 | 162.4 | 255 | 92.7 |
| 5.00 | 158.5 | 255 | 96.5 |

TABLE 2

| Thickness | Wedge | Background | Pixel |
|---|---|---|---|
| [mm] | [Px] | Contrast [Px] | Difference [Px] |
| 2.99 | 212.7 | 255 | 42.3 |
| 5.93 | 141.2 | 255 | 113.9 |
| 8.96 | 94.4 | 255 | 160.6 |
| 11.98 | 62.3 | 255 | 192.7 |
| 14.99 | 42.0 | 255 | 213.0 |
| 18.01 | 27.3 | 255 | 227.7 |
| 21.01 | 18.0 | 255 | 237.0 |
| 23.99 | 11.2 | 255 | 243.8 |
| 26.99 | 7.4 | 255 | 247.6 |
| 30.17 | 8.5 | 255 | 246.5 |

As illustrated in FIG. 8B, not all of the steps (e.g., a) in the first wedge image 530′ are differentiated from the background, thus, to ensure an accurate calibration, they were not included in Table 1. The Pixel difference [Px] can be graphed against the thickness of the aluminum wedge to create a calibration curve for each the first aluminum wedge, illustrated in FIG. 9A, and the second aluminum wedge, illustrated in FIG. 9B. As presented in Tables 1 and 2, the radiopacity of the aluminum wedges creates an exponential curve defining an asymptote at the background pixel content. Thus, seen in FIG. 9B as the second aluminum wedge increases in thickness, the Pixel difference [Px] approaches the background contrast, albeit at a decreasing rate. Thus, although the thickness is increasing, the aluminum wedge cannot block and/or scatter more x-rays. In this regard, to maximize efficiency, (e.g., cost, image production, etc.) the inventors have determined an optimal ratio of radiopaque powder to binder material, where adding additional radiopaque powder will provide diminishing returns in the equivalent aluminum thickness.

In each trial, multiple radiopaque components (some being attached to implantable devices) and a calibration means including a first aluminum wedge, a second aluminum wedge and a lead dot, were positioned to a backing paper. The calibration means were used to calibrate the pixel differences and generate calibration curves similar to those illustrated in FIGS. 9A-B.

FIGS. 10A-B, 11A-B, 12A-B, and 13A-B includes sample radiopaque components adjacent to a lead label number for identification in the x-ray image. FIG. 10A shows a digital image 600 comprising ten (10) samples, a first aluminum wedge 630, a second aluminum wedge 635, and a lead dot 640. The calibration tables for the first aluminum wedge 630 and the second aluminum wedge 635 FIG. 10A are provided above in Tables 1 and 2. An x-ray image was taken of each of the sample sets. The difference in the grayscale pixel density between the sample and the background was calculated to provide a measure of radiopacity. The pixel difference was then compared to the calibration curve for the respective aluminum wedges. Using the calibration curve, an equivalent thickness of aluminum was determined for each of the samples. Thus, the equivalent thickness of each sample, indicates the thickness of aluminum which produces the same contrast on the x-ray image. Table 3 shows a first sample set of radiopaque components and the equivalent thickness of each sample based on calibration Tables 1 & 2.

TABLE 3

| | | | | | Equivalent |
|---|---|---|---|---|---|
| | | | Background | Pixel | Aluminum |
| | | Sample | Contrast | Difference | Thickness |
| Sample | Description | [Px] | [Px] | [Px] | [mm] |
| 8a | Single Tantalum/silicone, 7 mm diameter × | 186.0 | 255.0 | 68.1 | 3.4 |
| | 0.3 mm thickness dot | | | | |
| 8b | Double Tantalum, two 7 mm diameter × | 166.1 | 255.0 | 88.9 | 4.4 |
| | 0.3 mm thickness dots (total thickness 0.6 mm) | | | | |
| 2 | Tantalum/silicone, two 7 mm diameter × | 194.6 | 255.0 | 60.5 | 3.1 |
| | 0.9 mm thickness dots (total thickness 1.8 mm) | | | | |
| 3 | Tantalum/silicone, 0.5 mm diameter rod | 215.9 | 255.0 | 39.1 | 2.3 |
| 9 | Tantalum/silicone, 0.1 mm thick × 12 cm | 126.8 | 255.0 | 128.3 | 4.0 |
| | length ribbon sealed to a graft | | | | |
| 10 | Tantalum/silicone, 0.1 mm thick × 12 cm | 180.7 | 255.0 | 74.3 | 3.7 |
| | length ribbon sealed to a graft | | | | |
| 11 | Tantalum/silicone, 0.1 mm thick × 12 cm | 210.7 | 255.0 | 44.3 | 2.5 |
| | length ribbon sealed to a graft | | | | |
| 12 | Tantalum/silicone, 0.2 mm thick × 12 cm | 182.2 | 255.0 | 72.8 | 3.7 |
| | length ribbon sealed to a graft | | | | |
| 13 | Tantalum/silicone, 0.2 mm thick × 12 cm | 80.6 | 255.0 | 174.4 | 10.5 |
| | length ribbon sealed to a graft | | | | |
| 14 | Tantalum/silicone, 0.2 mm thick × 12 cm | 171.8 | 255.0 | 83.2 | 4.2 |
| | length sealed graft | | | | |

Each of the samples is described in Table 3. Sample 9-11 comprises a radiopaque ribbon component at a first thickness (0.1 mm), while samples 12-14 comprise the radiopaque ribbon component at a second thickness (0.2 mm). Each of samples 9-11 comprise the same radiopaque component, other than the thickness thereof, applied to the same implantable device using the methods described above. The radiopaque component was applied both vertically and horizontally to each of the implantable devices. Table 3 and FIG. 10B illustrate the difference in the equivalent thickness of the radiopaque component when positioned about different areas of the implantable device. FIG. 10B shows an x-ray image 600′ of the digital image 600, and corresponding images of the first aluminum wedge 630′ the second aluminum wedge 635′ the lead dot 640′ and each of the radiopaque components. Notably, example dots (2, 8a and 8b) made in accordance with various embodiments described herein showed good visibility in the x-ray image. Additionally, example ribbons (9, 10, 12, 13, and 14) made in accordance with various embodiments described herein also showed good visibility in the x-ray image.

Thus, the inventive single tantalum dot having a thickness of 0.3 mm was, surprisingly, equivalent in radiopacity to an aluminum sample having a thickness of 3.4 mm. Similarly, when two of the inventive tantalum dots were stacked, each defining a thickness of 0.3 mm, for a total thickness of 0.6 mm, was, surprisingly, equivalent in radiopacity to an aluminum sample having a thickness of 4.4 mm. The inventive 0.5 mm diameter rod was, surprisingly, equivalent in radiopacity to an aluminum sample having a thickness of 2.3 mm.

### Example 2

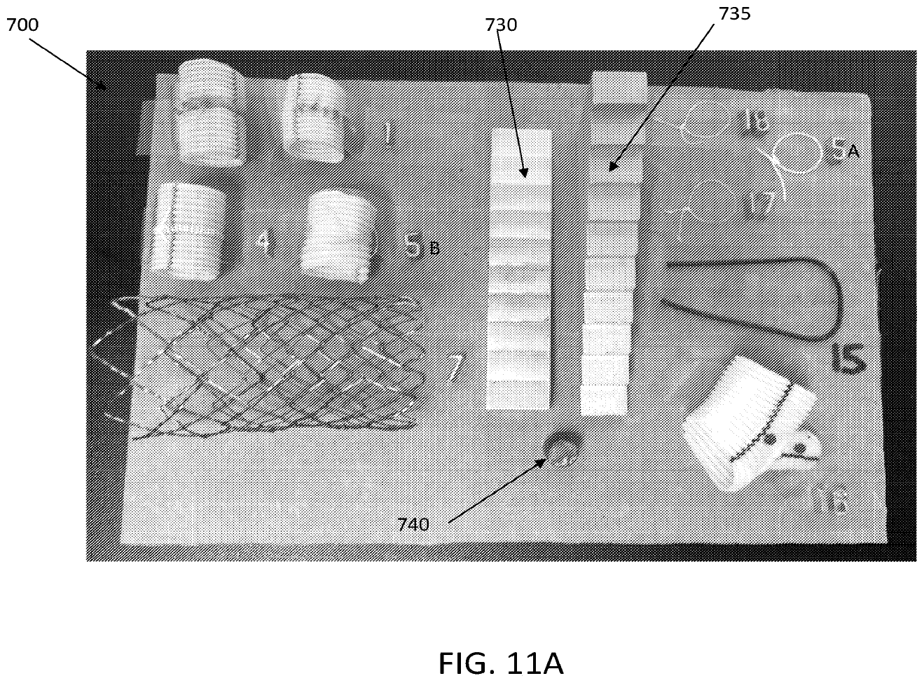

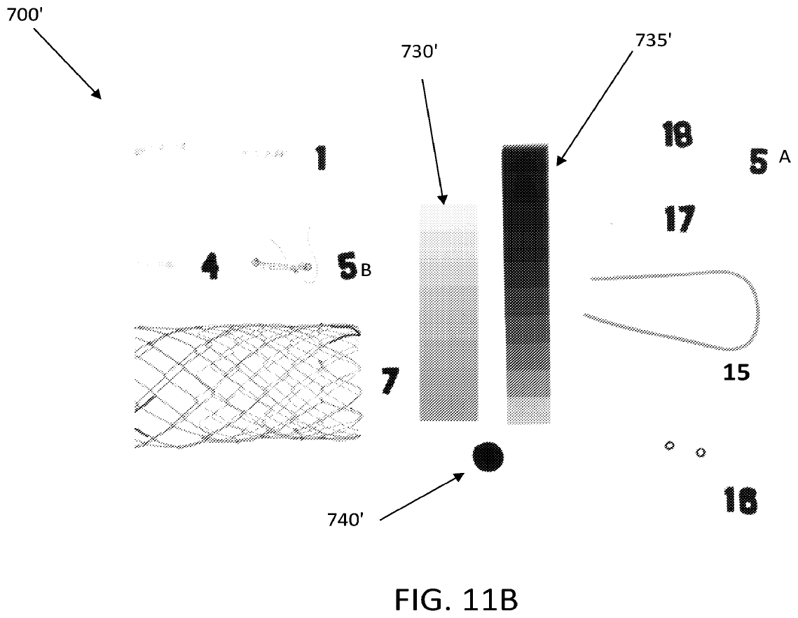

Similarly, FIG. 11A illustrates a digital image 700, corresponding to an x-ray image 700′ illustrated in FIG. 11B. FIG. 11A includes nine (9) samples, a first aluminum wedge 730, a second aluminum wedge 735, and a lead dot 740. The calibration table for the first aluminum wedge 730 is shown below in Table 4, and the calibration table for the second aluminum wedge 735 is shown below in Table 5. Although calibration tables corresponding to the digital image 600 presented in Tables 1-2 are similar to the calibration tables for the digital image 700 presented in Tables 4-5 slight differences appear, thus, emphasizing the importance of creating a calibration table for each digital image/x-ray image pair(s).

TABLE 4

| Thickness | Wedge | Background | Pixel |
|---|---|---|---|
| [mm] | [Px] | Contrast [Px] | Difference [Px] |
| 1.57 | 238.9 | 255 | 16.1 |
| 2.03 | 219.0 | 255 | 36.0 |
| 2.50 | 203.5 | 255 | 51.5 |
| 3.05 | 186.5 | 255 | 68.5 |
| 3.57 | 172.9 | 255 | 82.1 |
| 4.10 | 159.5 | 255 | 95.5 |
| 4.59 | 149.2 | 255 | 105.8 |
| 5.00 | 143.0 | 255 | 112.0 |

TABLE 5

| Thickness | Wedge | Background | Pixel |
|---|---|---|---|
| [mm] | [Px] | Contrast [Px] | Difference [Px] |
| 2.99 | 189.6 | 255 | 65.4 |
| 5.93 | 127.0 | 255 | 128.0 |
| 8.96 | 85.9 | 255 | 169.1 |
| 11.98 | 58.8 | 255 | 196.2 |
| 14.99 | 40.1 | 255 | 214.9 |
| 18.01 | 27.2 | 255 | 227.8 |
| 21.01 | 18.9 | 255 | 236.1 |
| 23.99 | 13.9 | 255 | 241.1 |
| 26.99 | 10.80 | 255 | 244.2 |
| 30.17 | 11.54 | 255 | 243.5 |

Each of the inventive samples are presented below in Table 6. An x-ray image 700′ was taken of the digital image 700, and using the first aluminum wedge 730′ and the second aluminum wedge 735′, calibration of an equivalent thickness was determined for each of the samples. As seen in FIG. 11B, the BaSO₄ suture was not visible under the imaging conditions. The gold wire (5B), the stent with gold marker (7), and the commercial crimped graft marker (16) appear with the greatest contrast to the background, and thus provide a greater equivalent thickness. Notably, however, an example rod (15) made in accordance with various embodiments described herein was very visible in the x-ray image. Also, an example suture (1) made in accordance with various embodiments described herein showed reasonably good visualization on the x-ray image.

TABLE 6

| | | | Background | Pixel | Equivalent |
|---|---|---|---|---|---|
| | | Sample | Contrast | Difference | Thickness |
| Sample | Description | [Px] | [Px] | [Px] | [mm] |
| 1 | Tantalum/PET suture sealed graft | 176.0 | 255.0 | 79.0 | 3.5 |
| 4 | BaSO₄ suture sealed graft | 206.4 | 255.0 | 48.6 | 2.4 |
| 5a | BaSO₄ suture | N/A | | | |
| 5b | Gold wire 0.003 diameter × 8 in length sealed | 64.6 | 255.0 | 190.4 | 13.8 |
| | within a graft | | | | |
| 7 | Stent with gold markers | 47.2 | 255.0 | 207.8 | 16.0 |
| 15 | Tantalum/silicone 1 mm diameter rod | 147.1 | 255.0 | 107.9 | 4.7 |
| 16 | Commercial crimped thoracoabdominal graft | 33.8 | 255.0 | 211.2 | 17.7 |
| | with radiopaque markers | | | | |
| 17 | 4823.50 G RevA | 218.0 | 255.0 | 37.0 | 2.1 |
| 18 | 4826.50 RevA | 212.7 | 255.0 | 42.3 | 2.2 |

The tantalum suture of sample 1 comprised a 0.25 mm diameter rod, braided on its exterior with 8 threads of polyester and tied in a standard surgical suture knot (friction square knot with another square knot on top of the first knot). The inventive tantalum suture of sample 1 was, surprisingly, equivalent in radiopacity to an aluminum sample having a thickness of 3.5 mm. Similarly, the inventive tantalum silicone 1 mm diameter rod of sample 15 was, surprisingly, equivalent in radiopacity to an aluminum sample having a thickness of 4.7 mm.

### Example 3

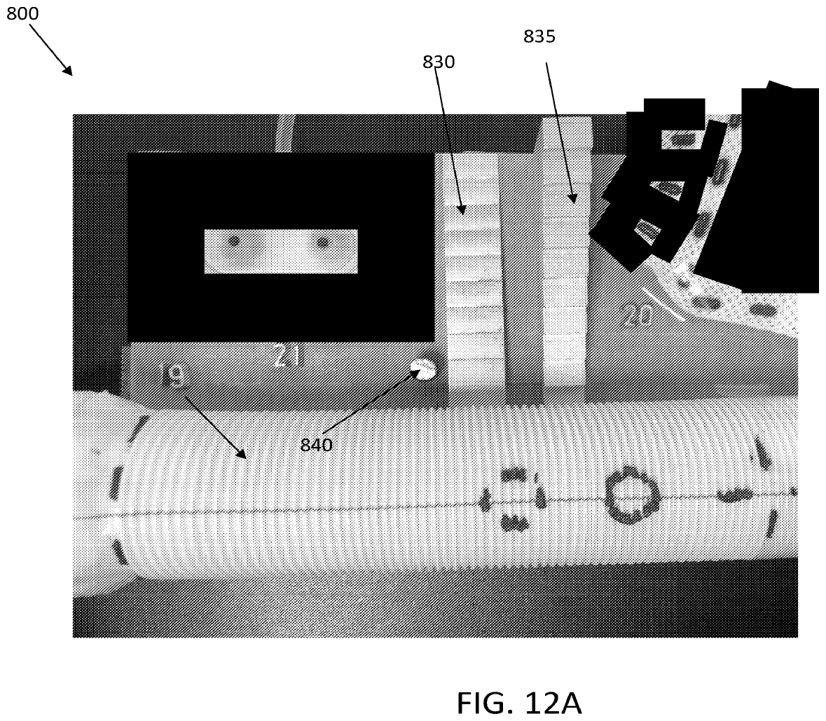

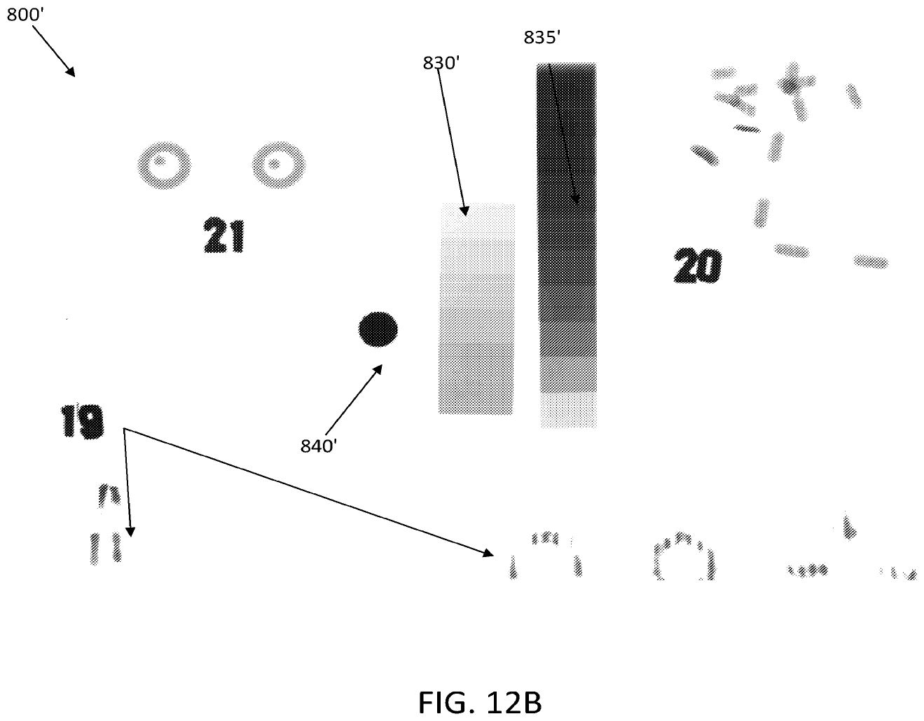

In another example, three (3) samples were compared for radiopacity. FIG. 12A illustrates a digital image 800 of the sample set. The digital image 800 includes a first aluminum wedge 830, a second aluminum wedge 835, and a lead dot 840 for calibration. The radiopaque material is positioned on and about a number of implantable devices. The calibration table for the first aluminum wedge 830 is presented in Table 7, and the calibration table for the second aluminum wedge 835 is presented in Table 8.

TABLE 7

| Thickness | Wedge | Background | Pixel |
|---|---|---|---|
| [mm] | [Px] | Contrast [Px] | Difference [Px] |
| 2.03 | 252.6 | 255 | 2.4 |
| 2.50 | 234.7 | 255 | 20.3 |
| 3.05 | 214.6 | 255 | 40.4 |
| 3.57 | 199.2 | 255 | 55.9 |
| 4.10 | 182.9 | 255 | 72.2 |
| 4.59 | 171.8 | 255 | 83.2 |
| 5.00 | 164.3 | 255 | 90.7 |

TABLE 8

| Thickness | Wedge | Background Contrast | Pixel Difference |
|---|---|---|---|
| [mm] | [Px] | [Px] | [Px] |
| 2.99 | 216.1 | 255 | 38.9 |
| 5.93 | 146.8 | 255 | 108.3 |
| 8.96 | 100.6 | 255 | 154.4 |
| 11.98 | 69.9 | 255 | 185.1 |
| 14.99 | 48.8 | 255 | 206.2 |
| 18.01 | 35.0 | 255 | 220.0 |
| 21.01 | 26.4 | 255 | 228.6 |
| 23.99 | 20.1 | 255 | 234.9 |
| 26.99 | 17.06 | 255 | 237.9 |
| 30.17 | 16.62 | 255 | 238.4 |

An x-ray image 800′ corresponding to the digital image 800 is presented in FIG. 12B. A description of each of the samples and the determined equivalent thickness is presented in Table 9. As illustrated in FIG. 12B each of the radiopaque components are visible in the x-ray image along with the image of the first aluminum wedge 830′ the second aluminum wedge 835′ and the lead dot 840′. When compared to the equivalent thicknesses of the gold suture, stent with a gold marker, and the Commercial crimped graft with marker, the radiopaque components illustrated in FIG. 12B have a much smaller equivalent thickness. Thus, a lower bound of an acceptable radiopacity equivalent thickness may be as low as 3.3 mm.

TABLE 9

| | | | Background | Pixel | Equivalent |
|---|---|---|---|---|---|
| | | Sample | Contrast | Difference | Thickness |
| Sample | Description | [Px] | [Px] | [Px] | [mm] |
| 21 | Device with radiopaque positioning tool | 176.0 | 255.0 | 79.0 | 4.5 |
| 19 | Valsalva with radiopaque markers | 206.4 | 255.0 | 48.6 | 3.3 |
| 20 | Device with radiopaque markers | 64.6 | 255.0 | 190.4 | 14.6 |

### Example 4

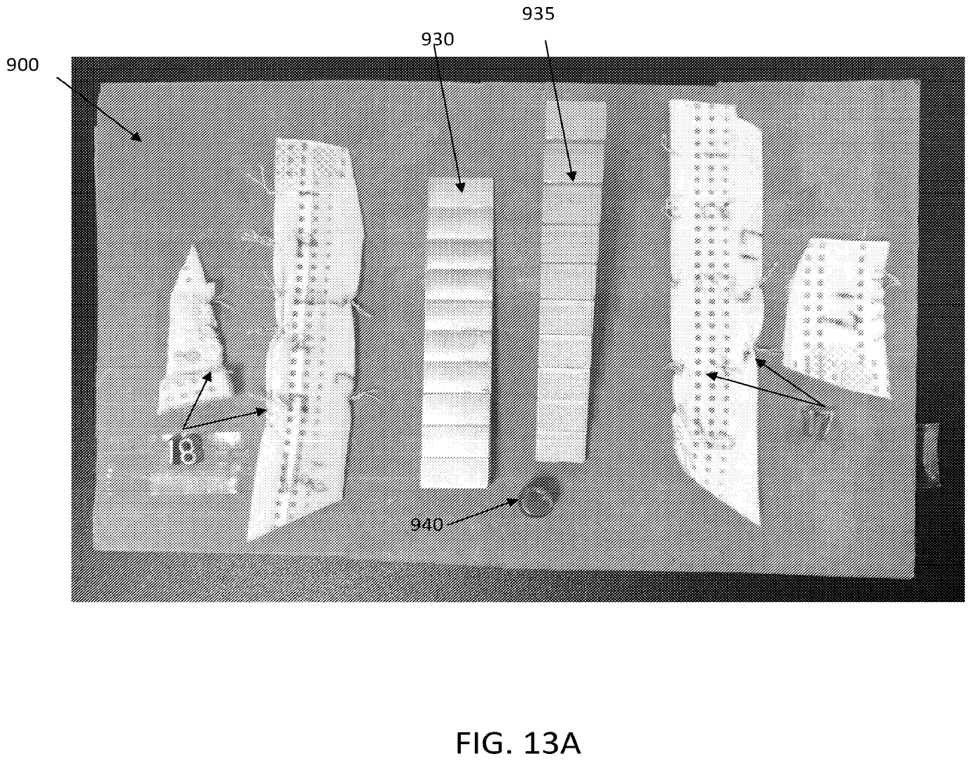

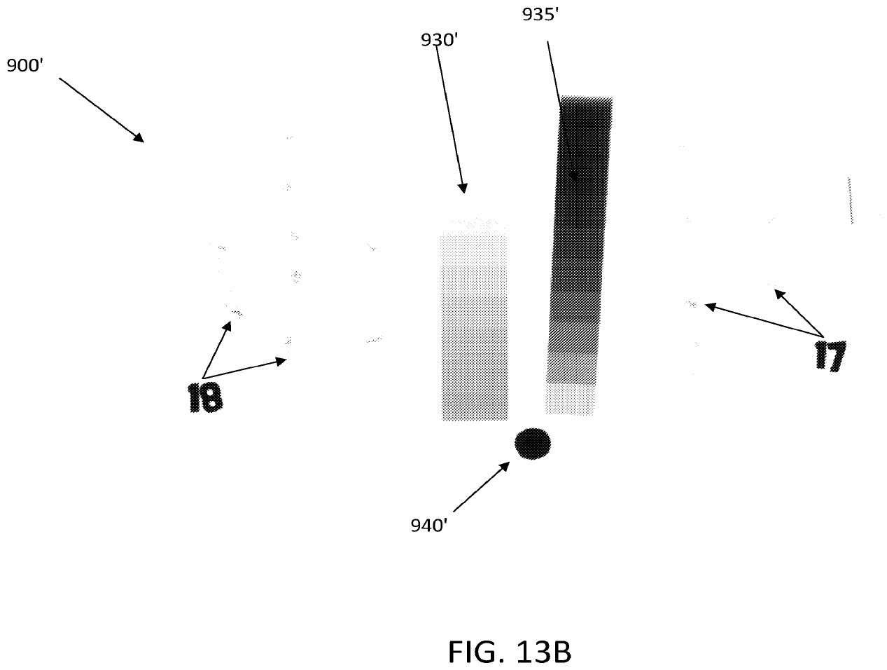

FIG. 13A illustrates two (2) samples, each comprising a plurality of sutures. The samples are depicted in a digital image 900 along with a first aluminum wedge 930, a second aluminum wedge 935, and a lead dot 940. The calibration table for the first aluminum wedge 930 is presented in Table 10. The calibration table for the second aluminum wedge 935 is not presented as the samples both fell within the range of the first aluminum wedge 930.

TABLE 10

| Thickness | Wedge | Background Contrast | Pixel Difference |
|---|---|---|---|
| [mm] | [Px] | [Px] | [Px] |
| 2.03 | 252.6 | 255 | 2.4 |
| 2.50 | 234.7 | 255 | 20.3 |
| 3.05 | 214.6 | 255 | 40.4 |
| 3.57 | 199.2 | 255 | 55.9 |
| 4.10 | 182.9 | 255 | 72.2 |
| 4.59 | 171.8 | 255 | 83.2 |
| 5.00 | 164.3 | 255 | 90.7 |

### Examples 5-8

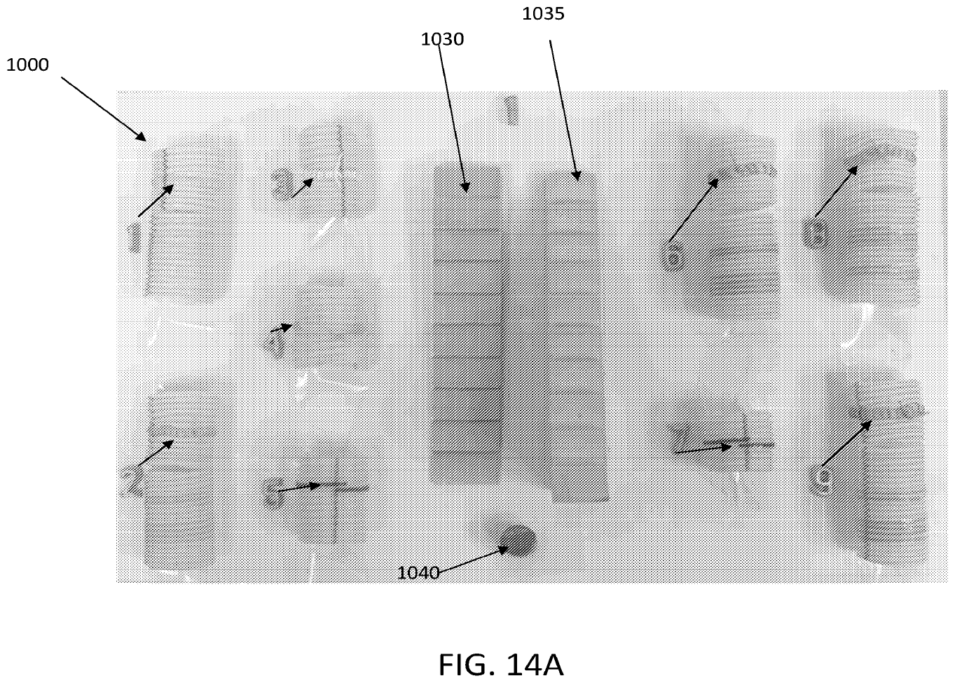

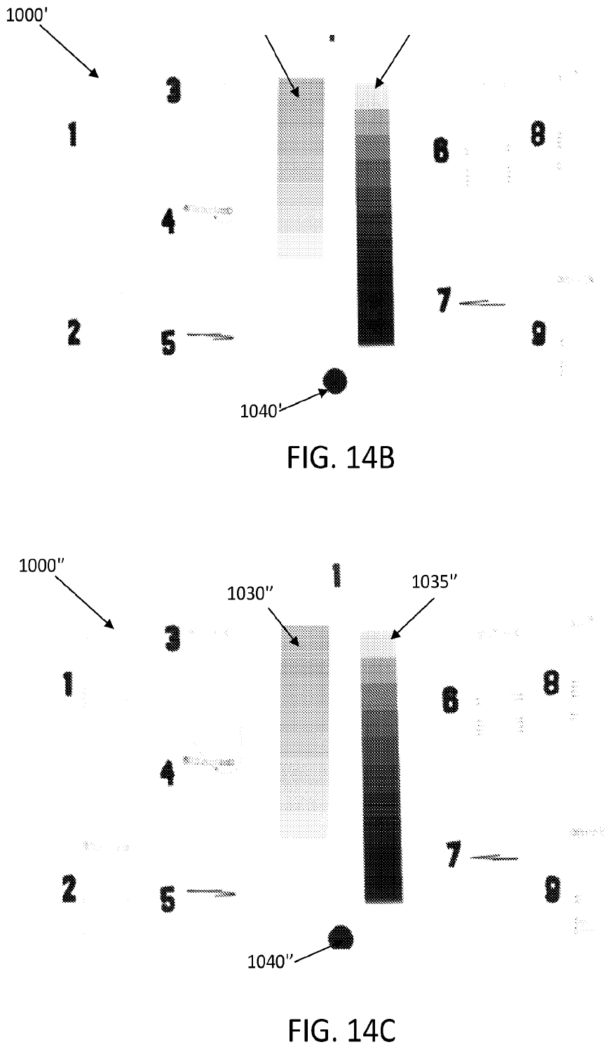

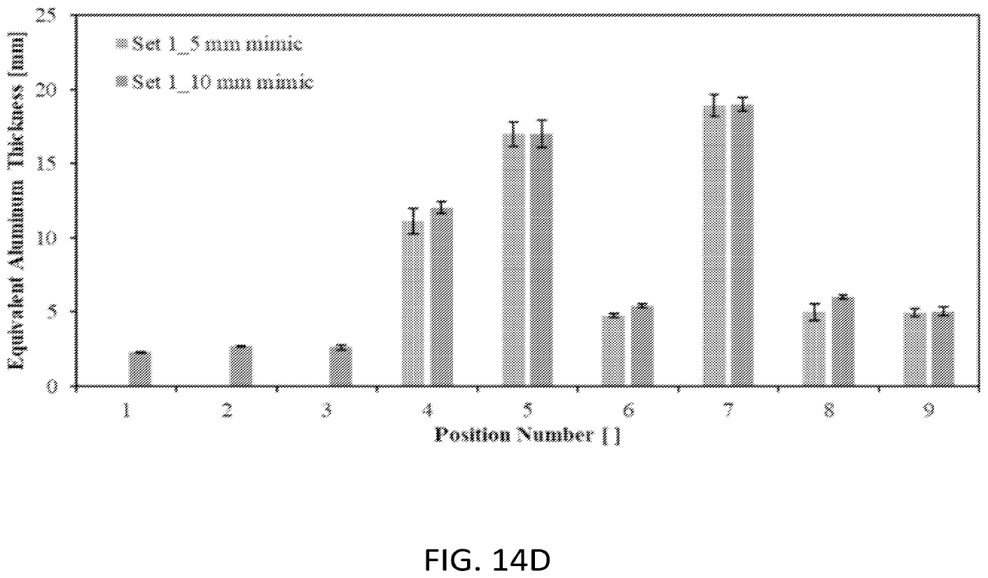

In these examples, the x-ray images were taken at more than one aluminum body thickness. The samples illustrated in FIGS. 14A-D, 15A-D, 16A-D, and 17A-D were imaged at an aluminum body thickness of 5 mm (B) and 10 mm (C), to compare the radiopacity equivalent thickness of each of the samples. Although the different body thickness did not change grayscale pixel density to a significant degree, a calibration table for each of the aluminum wedges, and equivalent thicknesses for each of the body thicknesses were calculated. Each of the graphs presented in FIGS. 14D, 15D, 16D, and 17D depict the average equivalent aluminum thickness for each sample, with the error bars representing plus/minus one standard deviation (n=3).

### Example 5

FIG. 14A shows a digital image 1000 comprising nine (9) samples including radiopaque sutures and radiopaque rods (e.g., 101 FIG. 1) affixed to implantable devices. The sample includes a first aluminum wedge 1030, a second aluminum wedge 1035, and a lead dot 1040 for calibration. The calibration table for each of the aluminum wedges at each of the body thicknesses are presented in Table 12.

TABLE 12

| | Step | Set 1_5 mm mimic | | | Set 1_10 mm mimic | | |
|---|---|---|---|---|---|---|---|
| | Thickness | Step | Background | Difference | Step | Background | Difference |
| Wedge | [mm] | [px] | [px] | [px] | [px] | [px] | [px] |
| 0.5 mm | 1.6 | 254 | 255 | 1 | 241 | 255 | 14 |
| steps | 2.0 | 239 | 255 | 16 | 229 | 255 | 26 |
| | 2.5 | 226 | 255 | 29 | 218 | 255 | 37 |
| | 3.1 | 211 | 255 | 44 | 206 | 255 | 49 |
| | 3.6 | 199 | 255 | 56 | 195 | 255 | 60 |
| | 4.1 | 187 | 255 | 68 | 185 | 255 | 70 |
| | 4.6 | 179 | 255 | 76 | 178 | 255 | 77 |
| | 5.0 | 175 | 255 | 80 | 176 | 255 | 79 |
| 3 mm | 3.0 | 231 | 255 | 24 | 224 | 255 | 31 |
| steps | 5.9 | 153 | 255 | 102 | 152 | 255 | 103 |
| | 9.0 | 102 | 255 | 153 | 104 | 255 | 151 |
| | 12.0 | 68 | 255 | 187 | 72 | 255 | 183 |
| | 15.0 | 45 | 255 | 210 | 49 | 255 | 206 |
| | 18.0 | 30 | 255 | 225 | 33 | 255 | 222 |
| | 21.0 | 19 | 255 | 236 | 22 | 255 | 233 |
| | 24.0 | 12 | 255 | 243 | 14 | 255 | 241 |
| | 27.0 | 8 | 255 | 247 | 10 | 255 | 245 |
| | 30.2 | 9 | 255 | 246 | 12 | 255 | 243 |

FIGS. 14B-C show a first x-ray image 1000′ taken at the first aluminum body thickness, and a second x-ray 1000″ taken at a second aluminum body thickness. As discussed, the change in the aluminum body thickness did not have a significant effect on the pixel difference of the samples. Each of the samples and the equivalent thickness is presented in Table 13. As depicted, the radiopaque component of samples 5 and 7 illustrate the position of the radiopaque component, the shape thereof, and the width thereof (with the correct scaled measurement). Notably, sample 1-5 includes 85% tantalum by weight and sample 1-7 includes 90% tantalum by weight.

TABLE 13

| | | 5 mm | 10 mm |
|---|---|---|---|
| | | Equivalent | Equivalent |
| | | Thickness | Thickness |
| Sample | Description | [mm] | [mm] |
| 1-1 | 887/152/N/DIN160 | N.D. | 2.3 ± 0.0 |
| 1-2 | P/875/0.15/B/D160 | N.D. | 2.7 ± 0.0 |
| 1-3 | Barium Sulfate suture on vascular graft | N.D. | 2.6 ± 0.0 |
| 1-4 | Gold suture on SSG vascular graft | 11.1 ± 0.9 | 12.0 ± 0.4 |
| 1-5 | 85% Ta/Si 0.5 mm rod on vascular graft | 17.0 ± 0.8 | 17.0 ± 1.0 |
| 1-6 | P/875/0.32/B/D160 | 4.7 ± 0.1 | 5.4 ± 0.1 |
| 1-7 | 90% Ta/Si 0.5 mm rod on vascular graft | 18.9 ± 0.8 | 19.0 ± 0.5 |
| 1-8 | P/EltexPPro/BiOCl 0.32 mm on vascular graft | 5.0 ± 0.5 | 6.0 ± 0.1 |
| 1-9 | P/EltexPPro/Bi₂O₂ on vascular graft | 5.0 ± 0.5 | 5.0 ± 0.3 |

As discussed above, each of the samples with an equivalent thickness above 4 is readily visible on the first and second x-ray images 1000′, 1000″. FIG. 14D depicts a graph of the average equivalent aluminum thickness for each sample, with the error bars representing plus/minus one standard deviation (n=3).

### Example 6

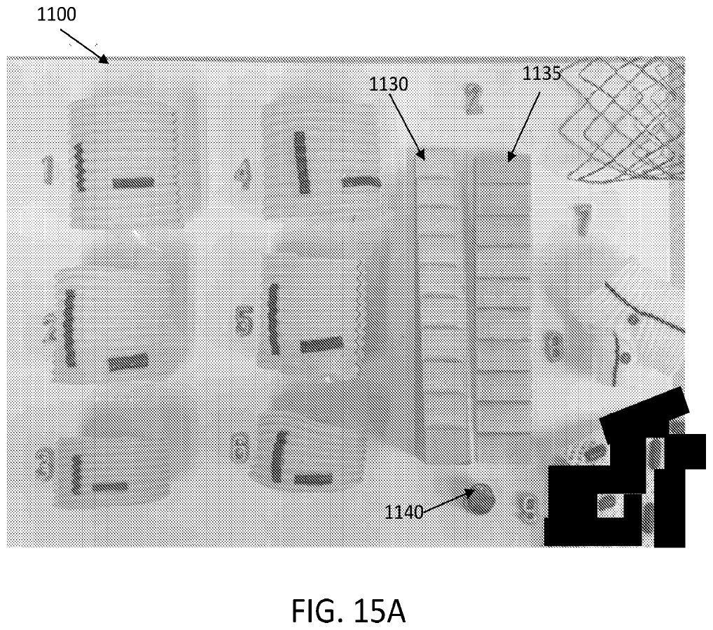

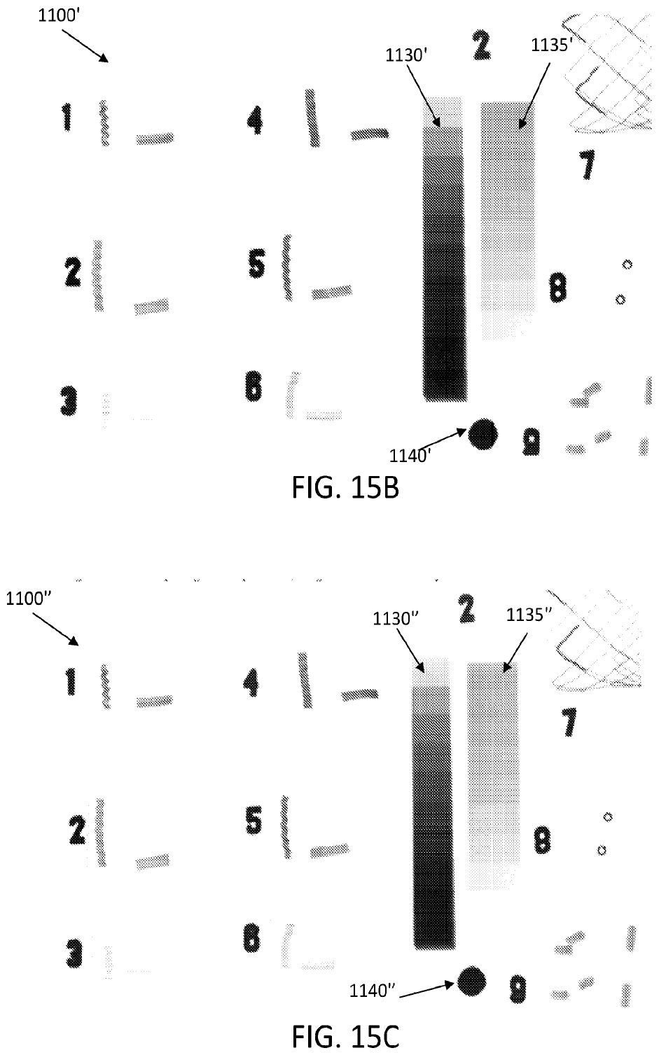

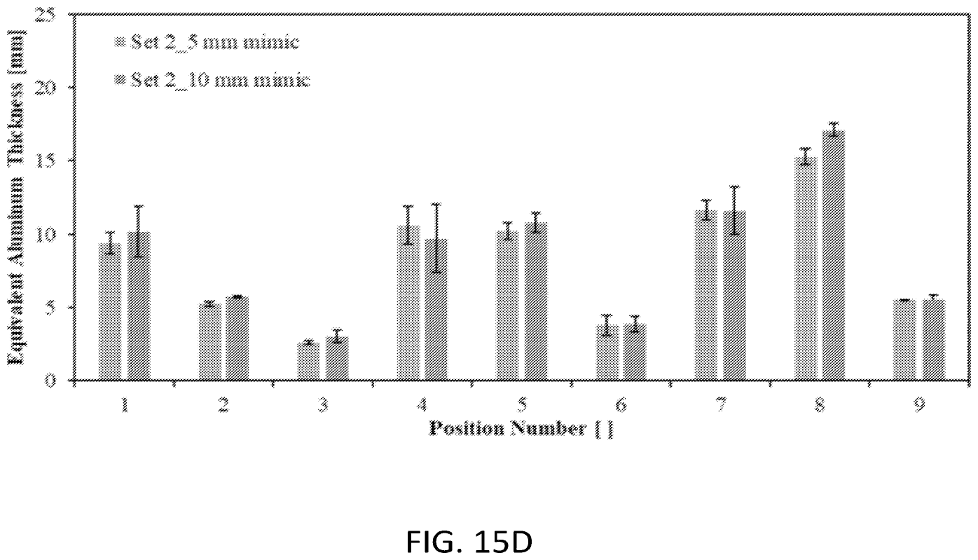

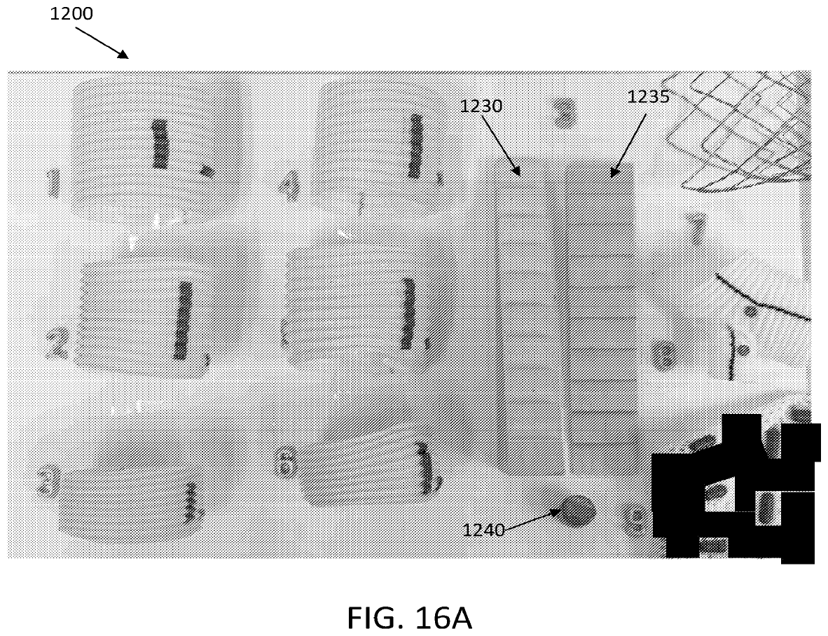

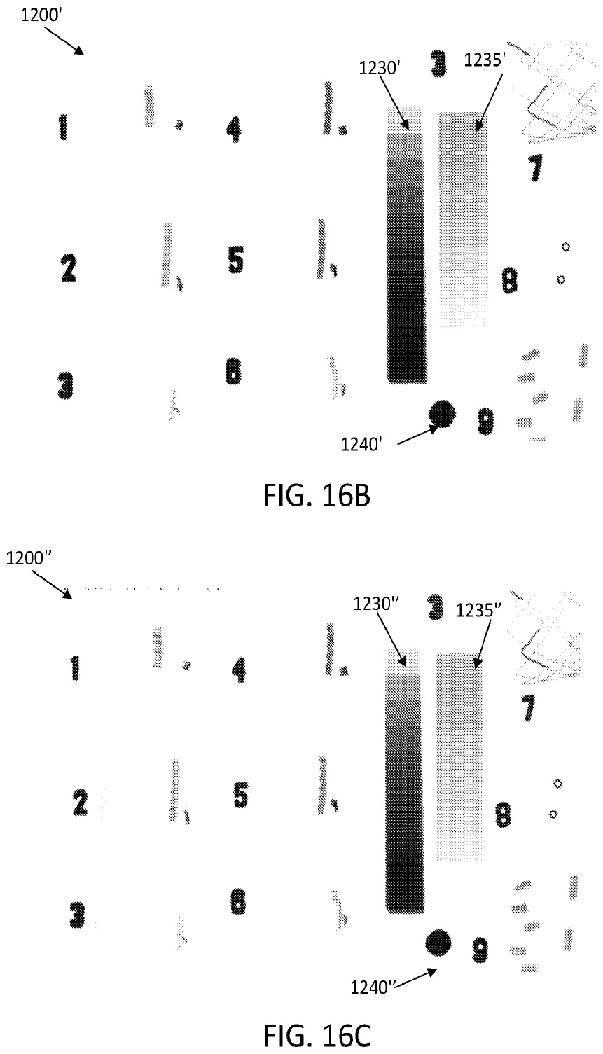

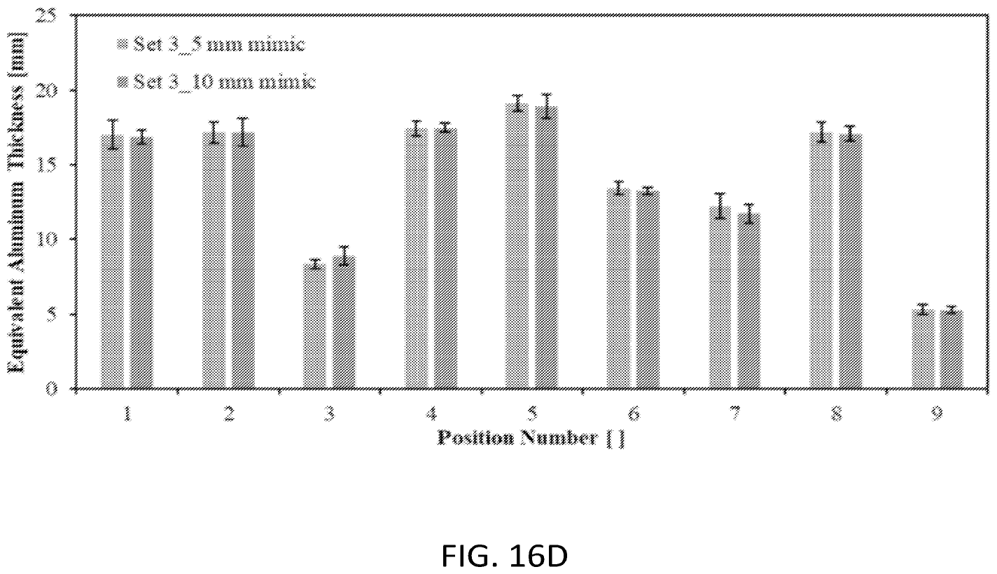

FIGS. 15A and 16A comprise the same nine (9) samples. The first six samples (2-1:2-6) and (3-1:3-6) are the same radiopaque component disposed on the implantable device rotated clockwise, demonstrating the effect of the location of the radiopaque component in relation to the x-ray on the equivalent thickness. Samples 7-9 are samples discussed previously that were identified as upper bounds (7-8) of equivalent aluminum thickness and average (9) equivalent aluminum thicknesses. Table 14 depicts a calibration table of a first aluminum wedge 1130 and a second aluminum wedge 1135 at each of the aluminum body thicknesses.

TABLE 14

| | Step | Set 2_5 mm mimic | | | Set 2_10 mm mimic | | |
|---|---|---|---|---|---|---|---|
| | Thickness | Step | Background | Difference | Step | Background | Difference |
| Wedge | [mm] | [px] | [px] | [px] | [px] | [px] | [px] |
| 0.5 mm | 1.6 | 246 | 255 | 9 | 245 | 255 | 10 |
| steps | 2.0 | 229 | 255 | 26 | 231 | 255 | 24 |
| | 2.5 | 216 | 255 | 39 | 220 | 255 | 35 |
| | 3.1 | 202 | 255 | 53 | 208 | 255 | 47 |
| | 3.6 | 190 | 255 | 65 | 197 | 255 | 58 |
| | 4.1 | 179 | 255 | 76 | 189 | 255 | 66 |
| | 4.6 | 171 | 255 | 84 | 183 | 255 | 72 |
| | 5.0 | 169 | 255 | 86 | 184 | 255 | 71 |
| 3 mm | 3.0 | 218 | 255 | 37 | 228 | 255 | 27 |
| steps | 5.9 | 145 | 255 | 110 | 157 | 255 | 98 |
| | 9.0 | 97 | 255 | 158 | 107 | 255 | 148 |
| | 12.0 | 65 | 255 | 190 | 74 | 255 | 181 |
| | 15.0 | 43 | 255 | 212 | 51 | 255 | 205 |
| | 18.0 | 29 | 255 | 226 | 35 | 255 | 220 |
| | 21.0 | 19 | 255 | 236 | 23 | 255 | 232 |
| | 24.0 | 12 | 255 | 243 | 15 | 255 | 240 |
| | 27.0 | 8 | 255 | 247 | 11 | 255 | 244 |
| | 30.2 | 7 | 255 | 248 | 13 | 255 | 242 |

Each of the samples depicted in the digital image 1100 shown in FIG. 15A are described in Table 15 with the corresponding equivalent thicknesses. As used herein, the percentages tantalum represent the percent, by weight, of tantalum in the tantalum/silicone mixture, before curing into the formed ribbon. Samples 2-1 through 2-6 are radiopaque components prepared according to various embodiments described herein, while samples 2-7 through 2-9 are used as baselines.

TABLE 15

| | | 5 mm | 10 mm |
|---|---|---|---|
| | | Equivalent | Equivalent |
| | | Thickness | Thickness |
| Sample | Description | [mm] | [mm] |
| 2-1 | 90% Tantalum/silicone, 0.1 mm ribbon on split skin graft | 9.4 ± 0.7 | 10.2 ± 1.7 |
| 2-2 | 85% Tantalum/silicone, 0.1 mm ribbon on split skin graft | 5.2 ± 0.2 | 5.8 ± 0.1 |
| 2-3 | 50% Tantalum/silicone 0.1 mm ribbon on split skin graft | 2.6 ± 0.1 | 3.0 ± 0.4 |
| 2-4 | 90% Tantalum/silicone, 0.2 mm ribbon on split skin graft | 10.6 ± 1.3 | 9.7 ± 2.3 |
| 2-5 | 85% Tantalum/silicone, 0.2 mm ribbon on split skin graft | 10.2 ± 0.6 | 10.8 ± 0.7 |
| 2-6 | 50% Tantalum/silicone, 0.2 mm ribbon on split skin graft | 3.8 ± 0.7 | 3.9 ± 0.5 |
| 2-7 | Stent w/gold marker | 11.6 ± 0.7 | 11.6 ± 1.6 |
| 2-8 | Commercial crimped graft w/markers | 15.3 ± 0.5 | 17.1 ± 0.4 |
| 2-9 | Device w/marker | 5.5 ± 0.0 | 5.5 ± 0.3 |

FIGS. 15B-C illustrate a first x-ray image 1100′ and a second x-ray image 1100″ taken at the first and second aluminum body thicknesses respectively. As seen in the images and depicted in Table 15 the body thickness did not have a large effect on the equivalent thickness of the radiopaque component. Notably, samples 2-1 (90% tantalum by weight) and 2-2 (85% tantalum by weight) are easily visible in both the first x-ray image 1100′ and the second x-ray image 1100″ as compared to sample 2-3 (50% tantalum by weight). Their radiopacity values reflect this. Similar comparisons are true when the thickness is increased to 0.2 mm for samples 2-4, 2-5, and 2-6. However, sample 2-5 at 50% tantalum by weight with a thickness of 0.2 mm is readily visible in the x-ray images and provides an acceptable radiopacity. One might expect that a 0.2 mm ribbon comprising 50% tantalum by weight, would have a radiopacity which is double that of a 0.1 mm ribbon comprising 50% tantalum by weight. However, this did not prove to be true, as shown above. Similarly, one might expect that increasing the percentage, by weight, of tantalum in a sample, may increase the radiopacity of the component by an equivalent percentage (e.g., 50% to 80% may show a 30% increase in radiopacity). However, this also proved not to be true as shown in Table 15, as increasing the percentage, by weight, of tantalum in the samples above provided an exponential increase in radiopacity. The inventors have surprisingly discovered herein that increasing the percent tantalum by weight has a far more significant effect on radiopacity than does increasing the thickness of a component.

FIG. 15D depicts a graph of the average equivalent aluminum thickness for each sample, with the error bars representing plus/minus one standard deviation (n=3). FIGS. 16A-D illustrate a second image 1200 of the samples described with relation to FIGS. 15A-D, where samples 1-6 are rotated clockwise. The rotation changes the orientation of the horizontal radiopaque component such that less of the component is exposed to the x-rays.

### Example 7

Table 16 depicts a calibration table of a first aluminum wedge 1230 and a second aluminum wedge 1235 at each of the aluminum body thicknesses.

TABLE 16

| | Step | Set 3_5 mm mimic | | | Set 3_10 mm mimic | | |
|---|---|---|---|---|---|---|---|
| | Thickness | Step | Background | Difference | Step | Background | Difference |
| Wedge | [mm] | [px] | [px] | [px] | [px] | [px] | [px] |
| 0.5 mm | 1.6 | 245 | 255 | 10 | 242 | 255 | 13 |
| steps | 2.0 | 230 | 255 | 25 | 227 | 255 | 28 |
| | 2.5 | 217 | 255 | 38 | 216 | 255 | 39 |
| | 3.1 | 204 | 255 | 51 | 206 | 255 | 49 |
| | 3.6 | 192 | 255 | 63 | 195 | 255 | 60 |
| | 4.1 | 181 | 255 | 74 | 186 | 255 | 69 |
| | 4.6 | 173 | 255 | 82 | 182 | 255 | 73 |
| | 5.0 | 171 | 255 | 84 | 181 | 255 | 74 |
| 3 mm | 3.0 | 221 | 255 | 34 | 226 | 255 | 29 |
| steps | 5.9 | 147 | 255 | 108 | 155 | 255 | 100 |
| | 9.0 | 99 | 255 | 156 | 106 | 255 | 149 |
| | 12.0 | 66 | 255 | 189 | 74 | 255 | 181 |
| | 15.0 | 44 | 255 | 211 | 50 | 255 | 205 |
| | 18.0 | 29 | 255 | 226 | 34 | 255 | 221 |
| | 21.0 | 19 | 255 | 236 | 23 | 255 | 232 |
| | 24.0 | 12 | 255 | 243 | 15 | 255 | 240 |
| | 27.0 | 7 | 255 | 248 | 10 | 255 | 245 |
| | 30.2 | 7 | 255 | 248 | 11 | 255 | 244 |

Each of the samples depicted in the digital image 1200 shown in FIG. 16A are described in Table 17 with the corresponding equivalent thicknesses. Samples 3-1 through 3-6 are the same radiopaque components as samples 2-1 through 2-6 illustrated in FIGS. 15A-C and depicted in Table 15, while samples 3-7 through 3-9 are used as baselines. FIGS. 16B-C illustrate a first x-ray image 1200′ and a second x-ray image 1200″ taken at the first and second aluminum body thicknesses respectively.

TABLE 17

| | | 5 mm | 10 mm |
|---|---|---|---|
| | | Equivalent | Equivalent |
| | | Thickness | Thickness |
| Sample | Description | [mm] | [mm] |
| 3-1 | 90% Tantalum/silicone, 0.1 mm ribbon on split skin graft | 17.0 ± 1.0 | 16.9 ± 0.4 |
| 3-2 | 85% Tantalum/silicone, 0.1 mm ribbon on split skin graft | 17.2 ± 0.7 | 17.2 ± 0.9 |
| 3-3 | 50% Tantalum/silicone, 0.1 mm ribbon on split skin graft | 8.4 ± 0.3 | 8.9 ± 0.6 |
| 3-4 | 90% Tantalum/silicone, 0.2 mm ribbon on split skin graft | 17.4 ± 0.5 | 17.5 ± 0.3 |
| 3-5 | 85% Tantalum/silicone, 0.2 mm ribbon on split skin graft | 19.1 ± 0.5 | 18.9 ± 0.8 |
| 3-6 | 50% Tantalum/silicone, 0.2 mm ribbon on split skin graft | 13.4 ± 0.4 | 13.3 ± 0.2 |
| 3-7 | Stent w/gold marker | 12.2 ± 0.8 | 11.7 ± 0.6 |
| 3-8 | Commercial crimped graft w/markers | 17.2 ± 0.7 | 17.1 ± 0.5 |
| 3-9 | Device w/marker | 5.3 ± 0.3 | 5.3 ± 0.2 |

In comparing the samples in Table 15 and Table 17, orientation of the radiopaque component may be the cause of the difference in equivalent thicknesses. The radiopaque components, for example, may have been disposed such that the thickness of the component, rather than length or width, was facing the x-ray in Table 17. For example, sample 2-1, at an aluminum body thickness of 5 mm has an equivalent thickness 9.4±0.7. In comparison, sample 3-1, the same sample at the same aluminum body thickness, has an equivalent thickness of 17.0±1.0. Without wanting to be bound by theory, the amount of surface area of the radiopaque component exposed to the x-rays may contribute to the difference in the equivalent thickness shown when comparing the two sample sets. FIG. 16D depicts a graph of the average equivalent aluminum thickness for each sample, with the error bars representing plus/minus one standard deviation (n=3).

### Example 8

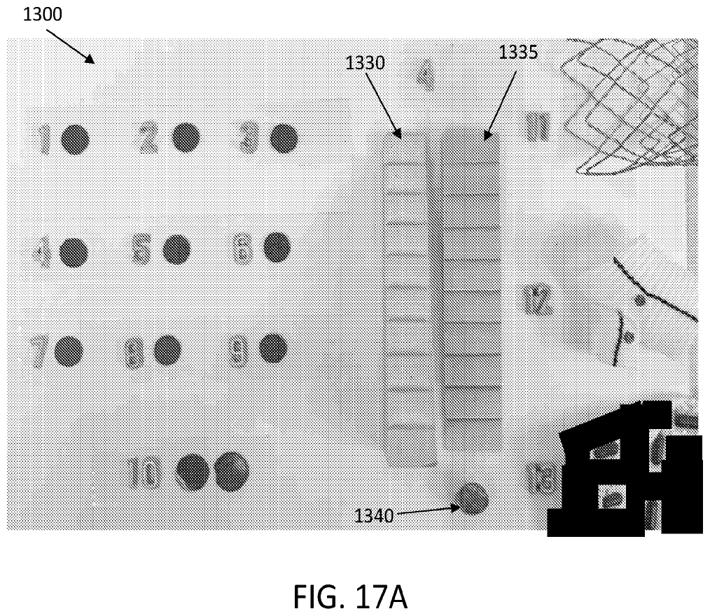

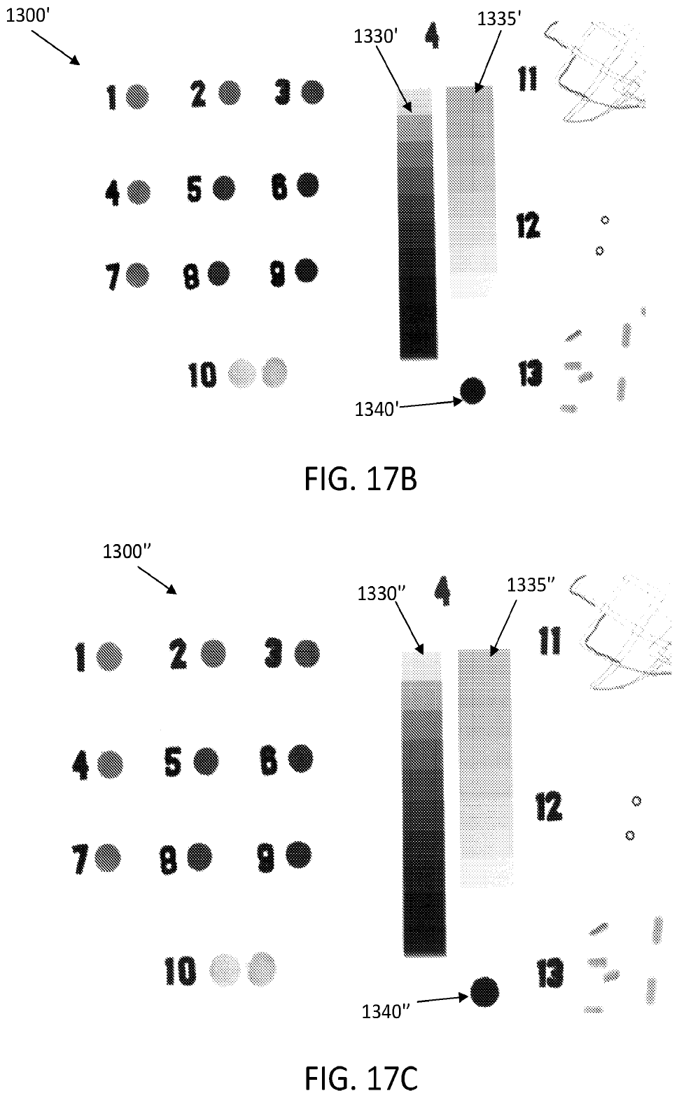

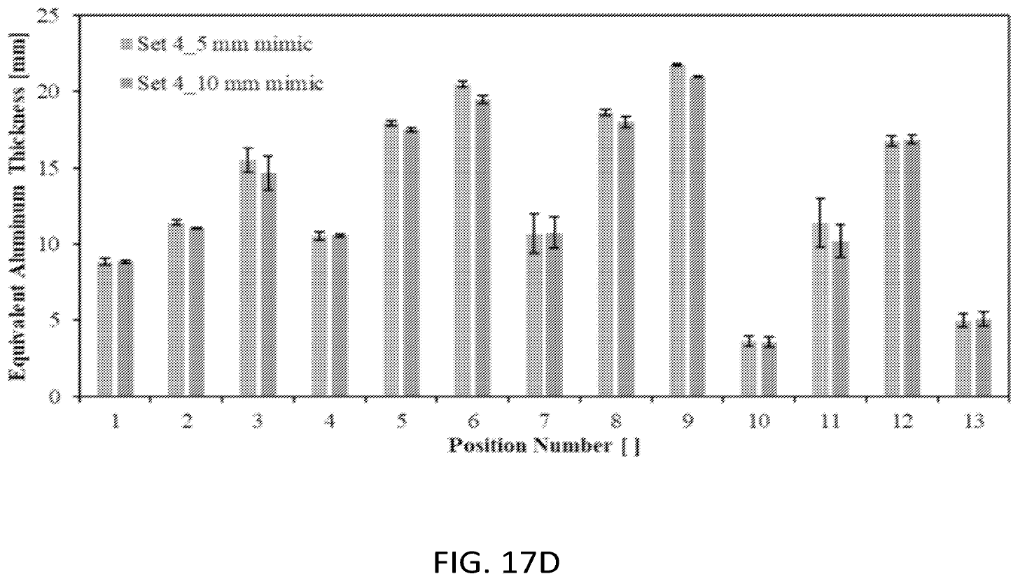

FIG. 17A shows a digital image 1300 depicting a plurality of radiopaque component dots (e.g., 103 FIG. 3A). The compositions of each of the samples is presented in Table 19, while Table 18 depicts a calibration table of a first aluminum wedge 1330 and a second aluminum wedge 1335 at each of the aluminum body thicknesses. As discussed herein, samples 4-11 through 4-13 correspond to samples 2-7 through 2-9.

TABLE 18

| | Step | Set 4_5 mm mimic | | | Set 4_10 mm mimic | | |
|---|---|---|---|---|---|---|---|
| | Thickness | Step | Background | Difference | Step | Background | Difference |
| Wedge | [mm] | [px] | [px] | [px] | [px] | [px] | [px] |
| 0.5 mm | 1.6 | 244 | 255 | 11 | 243 | 255 | 12 |
| steps | 2.0 | 228 | 255 | 27 | 229 | 255 | 26 |
| | 2.5 | 215 | 255 | 40 | 217 | 255 | 38 |
| | 3.1 | 200 | 255 | 55 | 205 | 255 | 50 |
| | 3.6 | 190 | 255 | 65 | 195 | 255 | 60 |
| | 4.1 | 179 | 255 | 76 | 185 | 255 | 70 |
| | 4.6 | 172 | 255 | 83 | 180 | 255 | 75 |
| | 5.0 | 168 | 255 | 87 | 179 | 255 | 76 |
| 3 mm | 3.0 | 218 | 255 | 37 | 225 | 255 | 30 |
| steps | 5.9 | 145 | 255 | 110 | 155 | 255 | 100 |
| | 9.0 | 97 | 255 | 158 | 106 | 255 | 149 |
| | 12.0 | 64 | 255 | 191 | 72 | 255 | 183 |
| | 15.0 | 43 | 255 | 212 | 49 | 255 | 206 |
| | 18.0 | 28 | 255 | 227 | 33 | 255 | 222 |
| | 21.0 | 18 | 255 | 237 | 22 | 255 | 233 |
| | 24.0 | 11 | 255 | 244 | 14 | 255 | 241 |
| | 27.0 | 8 | 255 | 247 | 10 | 255 | 245 |
| | 30.2 | 7 | 255 | 248 | 0 | 255 | 255 |

TABLE 19

| | | 5 mm | 10 mm |
|---|---|---|---|
| | | Equivalent | Equivalent |
| | | Thickness | Thickness |
| Sample | Description | [mm] | [mm] |
| 4-1 | 85% Tantalum/Si 6 mm diameter × 0.4 mm thickness dot | 8.8 ± 0.2 | 8.8 ± 0.1 |
| 4-2 | 85% Tantalum/Si 6 mm diameter × 0.6 mm thickness dot | 11.4 ± 0.2 | 11.0 ± 0.0 |
| 4-3 | 85% Tantalum/Si 6 mm diameter × 0.8 mm thickness dot | 15.5 ± 0.8 | 14.6 ± 1.1 |
| 4-4 | 90% Tantalum/Si 6 mm diameter × 0.3 mm thickness dot | 10.5 ± 0.3 | 10.6 ± 0.1 |
| 4-5 | 90% Tantalum/Si 6 mm diameter × 0.6 mm thickness dot | 17.9 ± 0.1 | 17.5 ± 0.1 |
| 4-6 | 90% Tantalum/Si 6 mm diameter × 0.9 mm thickness dot | 20.5 ± 0.2 | 19.5 ± 0.3 |
| 4-7 | 94.4% Tantalum/Si 6 mm diameter × 0.3 mm thickness dot | 10.7 ± 1.3 | 10.7 ± 1.0 |
| 4-8 | 94.4% Tantalum/Si 6 mm diameter × 0.6 mm thickness dot | 18.6 ± 0.2 | 18.0 ± 0.4 |
| 4-9 | 94.4% Tantalum/Si 6 mm diameter × 0.8.5 mm thickness dot | 21.7 ± 0.1 | 21.0 ± 1.0 |
| 4-10 | 50% Tantalum/Si dot | 3.6 ± 0.3 | 3.6 ± 0.3 |
| 4-11 | Stent w/gold marker | 11.4 ± 1.6 | 10.2 ± 1.1 |
| 4-12 | Commercial crimped graft w/markers | 16.8 ± 0.3 | 16.9 ± 0.3 |
| 4-13 | Device w/marker | 5.0 ± 0.4 | 5.1 ± 0.4 |

FIGS. 17B-C illustrate a first x-ray image 1300′ and a second x-ray image 1300″ taken at the first and second aluminum body thicknesses respectively. As can be seen in each of the first and second x-ray images 1300′, 1300″ and Table 19 there is a significant increase in equivalent thickness when the radiopaque material is increased from 85% to 90%. There is a smaller increase in equivalent thickness when the radiopaque material is increased from 90% to 94%, regardless of thickness of the radiopaque component. Each of the samples comprising 85%, 90%, and 94%, respectively, of the radiopaque material are above a baseline equivalent thickness (e.g., 5.0 thickness of sample 4-13—although other base line equivalent thicknesses are contemplated). It is also worth noting that sample 4-10 is a dot with 50% tantalum by weight still shows up reasonably well on the x-ray images, providing an acceptable radiopacity. FIG. 17D depicts a graph of the average equivalent aluminum thickness for each sample, with the error bars representing plus/minus one standard deviation (n=3).

Of note, the radiopaque composition including tantalum and silicone is visible under some, but not all medical images. For example, FIG. 18A illustrates an example implantable medical device 1410 comprising two radiopaque components 1401. FIGS. 18B-E illustrate example medical images which may be used prior to and/or during medical procedures. FIG. 18B illustrates a CT scan image depicting the medical device implanted into a body. In the CT scan the two radiopaque components 1401 are visible. Similarly, FIG. 18C illustrated a 3D CT scan image, which is a surface rendition of the CT scan. Here the image depicts the positioning of the implantable medical device and the radiopaque components 1401 on the organ which received the implantable medical device. FIG. 18D illustrates a fluoroscopy image (e.g., x-ray) of the body including the implantable medical device, depicting the position of the two radiopaque components 1401. Thus, as discussed above the radiopaque components are detectable by fluoroscopy. In contrast FIG. 18E illustrates a magnetic resonance imaging image of the body including the implantable medical device. Here the medical device including the two radiopaque components are not visible. In this regard, there are some radiopaque materials which do not show up on an MRI too brightly, and thus, will not occlude parts of the MRI. Additionally, due to the strong magnetic field of an MRI, some metals may vibrate, which could move and/or damage the metal. In testing there was no damage to either the implantable medical device or the radiopaque components after the MRI.

### Example 9

In this example, a radiopacity analysis was conducted per ASTM F640-23. Samples were mounted onto 4″ by 6″ cardstock sheets in various groups. Each paper card containing samples was placed on the detector of a Faxitron Ultrafocus digital x-ray system, along with aluminum step wedges (alloy 1100, 0.5 mm and 3 mm steps) and a 10 mm aluminum body mimic (placed underneath the paper card). The samples were imaged under the following x-ray settings: source target—tungsten (W), filter type—none, detector type—CMOS, source to detector distance—24.57 inches, object to detector distance—0 inches, imaging system resolution—3072×2048 pixels, peak voltage—70 kV, exposure—1.44 mAs. Digital images were collected and analyzed by measuring pixel intensity of the sample and background sample areas. Pixel intensity measurements of the samples were taken at three different regions. Background pixel intensity measurements were taken at the background position nearest to the related sample portion. Pixel intensity differences for the aluminum stepped wedges were calculated in the same manner and were used to generate an exponential calibration curve. Equivalent aluminum thickness for the samples was then calculated using the aluminum calibration curve. Three individual readings per sample were taken with three controls (background measurements) immediate adjacent to the sample image. The three replicates provide the mean and standard deviation reported here.

### Set #1

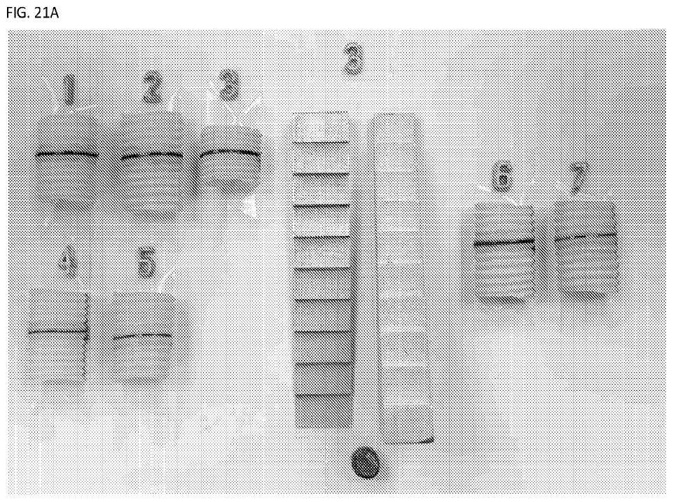

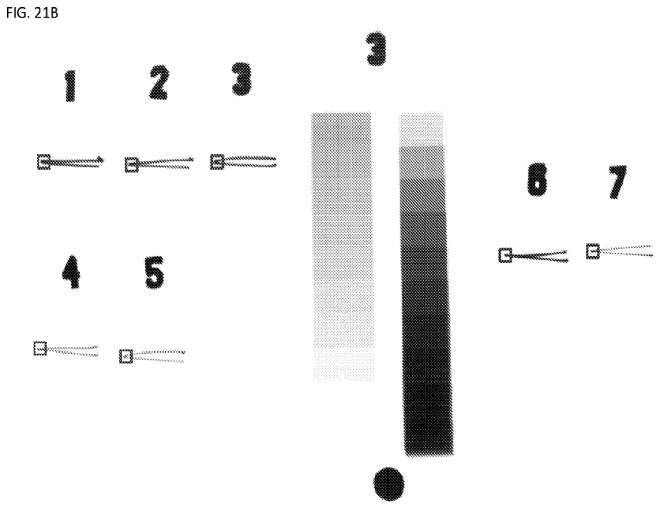

A camera image of Set #1 is shown in FIG. 19A (aluminum step wedges and lead ingot shown in the center) and the raw x-ray image of Set #1 is shown in FIG. 19B. For samples 1 and 2, sutures were wrapped about a 10 mm graft. In sample 1, ten individual sutures were wrapped and in sample 2, twelve individual sutures were wrapped. The sutures are provided in pairs, each pair having two thicknesses: 0.2 mm and 0.5 mm. Starting at the bottom of the graft, the pairs of samples increase in percent, by weight, of tantalum from 50% to 60% to 70% to 80% to 90% and for the spherical tantalum in sample 2, to 94%. As noted below, sample 3 is gold wire braided with PET that was used for comparison purposes. The equivalent aluminum thickness for samples 1-6 (Set #1) is shown in Table 21.

TABLE 21

| | | Equivalent |
|---|---|---|
| | | Aluminum |
| Sample # | Product Description | Thickness (mm) |
| 1 | Angular tantalum series 50%, 60%, 70%, 80%, | 20.9 ± 0.2 |
| | and 90%, each provided on a 10 mm graft | |
| 2 | Spherical Tantalum series 50%, 60%, 70%, 80%, | 19.5 ± 0.1 |
| | 90%, and 94%, each provided on a 10 mm graft | |
| 3 | Gold wire 0.003 × 8 inches sealed graft | 8.0 ± 0.6 |
| 4 | Running 3-0 tantalum suture on a 10 mm graft | 12.5 ± 1.6 |
| 5 | Knotted multiple 3-0 tantalum sutures on a | 18.5 ± 0.6 |
| | 10 mm graft | |
| 6 | Loop of 3-0 tantalum suture with a knot | 6.0 ± 0.2 |
| 7 | Sputter coated platinum braid | Not Detectible |

As can be seen, other than sample 6, each experimental tantalum sample (1-2, 4-5) provided an equivalent aluminum thickness of between 12.5 and 20.9 mm. An equivalent aluminum thickness of above 10 mm is acceptable for arterial or endoscopic placement. As such, each of samples 1, 2, 4, and 5 was found to be acceptable. Samples having equivalent aluminum thicknesses of above about 17 or 18 mm (samples 1, 2, and 5) were found to have excellent radiopacity.

### Set #2

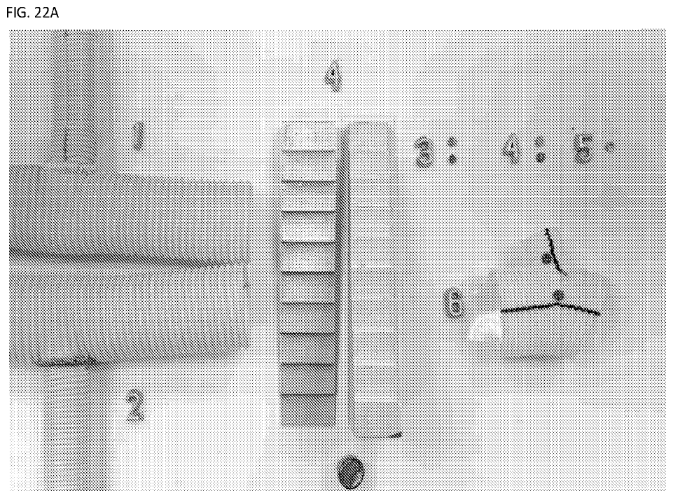

A camera image of Set #2 is shown in FIG. 20A (aluminum step wedges and lead ingot shown in the center) and the raw x-ray image of Set #2 is shown in FIG. 20B. In this Set #2, angular tantalum was compared to spherical tantalum at various weight percentages. The equivalent aluminum thickness for samples 1-10 (Set #2) is shown in Table 22.

TABLE 22

| | | Equivalent |
|---|---|---|
| | | Aluminum |
| Sample # | Product Description | Thickness (mm) |
| 1 | Angular tantalum 50%, 0.5 mm tantalum rod. | 13.6 ± 0.5 |
| 2 | Spherical Tantalum 50% 0.5 mm tantalum rod | 6.3 ± 0.3 |
| 3 | Angular tantalum 60%, 0.5 mm tantalum rod. | 17.5 ± 0.3 |
| 4 | Spherical Tantalum 60% 0.5 mm tantalum rod | 15.5 ± 0.4 |
| 5 | Angular tantalum 70%, 0.5 mm tantalum rod. | 16.5 ± 0.6 |
| 6 | Spherical Tantalum 70% 0.5 mm tantalum rod | 16.5 ± 0.4 |
| 7 | Angular tantalum 80%, 0.5 mm tantalum rod. | 17.9 ± 0.2 |
| 8 | Spherical Tantalum 80% 0.5 mm tantalum rod | 20.2 ± 0.4 |
| 9 | Angular tantalum 80%, 0.2 mm tantalum rod. | 12.4 ± 0.7 |
| 10 | Spherical Tantalum 80% 0.2 mm tantalum rod | 13.6 ± 0.5 |

As can be seen, at lower weight percentages of tantalum (50% to 60%), angular tantalum has a higher equivalent aluminum thickness than spherical tantalum. At 70% tantalum by weight, the samples performed substantially equivalently. At weight percentages of 80% or greater, regardless of thickness of the rod between 0.2 and 0.5 mm, spherical tantalum has a higher equivalent aluminum thickness than angular tantalum. Other than sample 2 (spherical tantalum, 50% by weight, 0.5 mm thickness), all samples showed acceptable results and many samples had excellent results.

### Set #3

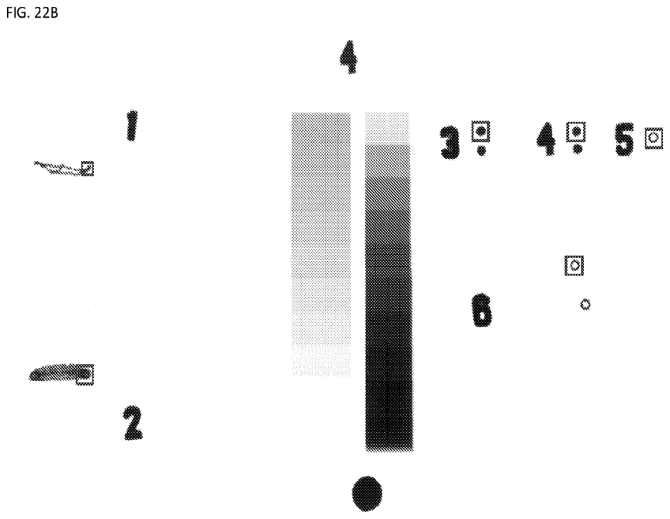

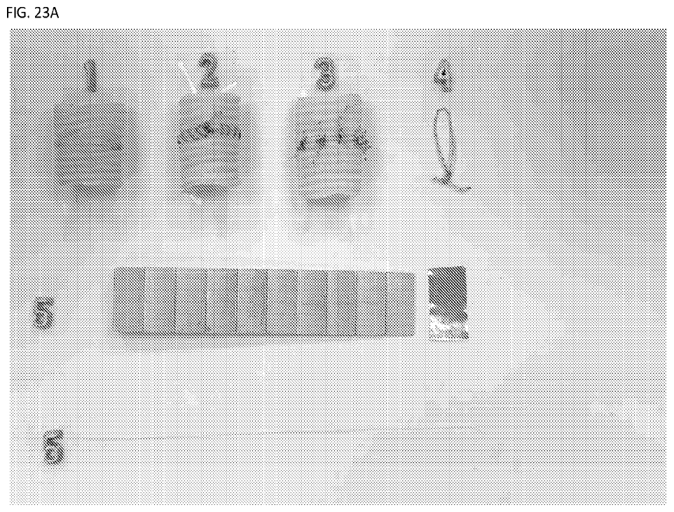

A camera image of Set #3 is shown in FIG. 21A (aluminum step wedges and lead ingot shown in the center) and the raw x-ray image of Set #3 is shown in FIG. 21B. In this Set #3, high weight percentages (90% to 94%) of tantalum were compared in rods of differing thicknesses and differing tantalum sources (angular versus spherical). The equivalent aluminum thickness for samples 1-7 (Set #3) is shown in Table 23.

TABLE 23

| | | Equivalent |
|---|---|---|
| | | Aluminum |
| Sample # | Product Description | Thickness (mm) |
| 1 | Angular tantalum 90%, 0.5 mm tantalum rod. | 19.6 ± 0.3 |
| 2 | Spherical Tantalum 90% 0.5 mm tantalum rod | 18.9 ± 0.4 |
| 3 | Angular 90% tantalum 0.5 mm rod on graft | 18.8 ± 0.2 |
| 4 | Angular tantalum 90%, 0.2 mm tantalum rod. | 16.0 ± 0.3 |
| 5 | Spherical Tantalum 90% 0.2 mm tantalum rod | 14.4 ± 0.5 |
| 6 | Spherical Tantalum 94% 0.5 mm tantalum rod | 19.3 ± 0.3 |
| 7 | Spherical Tantalum 94% 0.2 mm tantalum rod | 15.7 ± 0.3 |

As can be seen, at high weight percentages of tantalum (90% to 94%), angular tantalum and spherical tantalum provide acceptable and, in many cases, excellent radiopacity. The 0.2 mm rod provided acceptable radiopacity in all tests and the 0.5 mm rod provided excellent radiopacity in all tests.

### Set #4

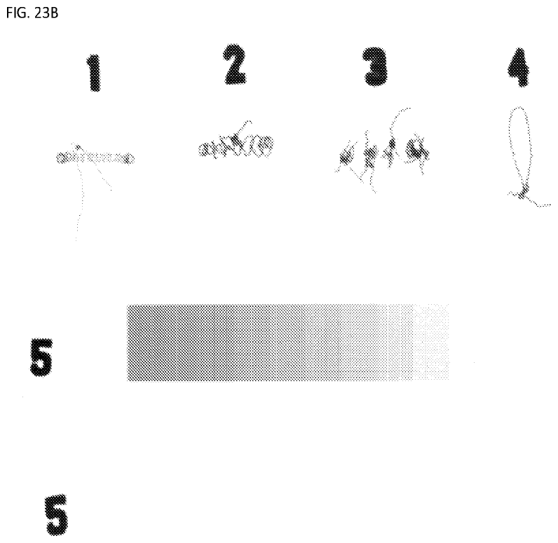

A camera image of Set #4 is shown in FIG. 22A (aluminum step wedges and lead ingot shown in the center) and the raw x-ray image of Set #4 is shown in FIG. 22B. In this Set #3, the inventors compared radiopaque materials of differing shapes and sizes. The equivalent aluminum thickness for samples 1-6 (Set #4) is shown in Table 24.

TABLE 24

| | | Equivalent |
|---|---|---|
| | | Aluminum |
| Sample # | Product Description | Thickness (mm) |
| 1 | Branched graft with an embedded 90%, 0.5 mm | 19.1 ± 0.4 |
| | tantalum rod | |
| 2 | Branched graft w/3-0, 0.2 mm tantalum suture | 21.4 ± 1.2 |
| | for cover stitch. | |
| 3 | Tantalum Sheet - 0.63 mm (0.025″)Thick | 19.9 ± 0.4 |
| 4 | Tantalum Sheet - 0.7 mm Thick | 20.1 ± 0.2 |
| 5 | Tantalum Tube - 1.232 mm ID, 0.4 mm Wall | 16.5 ± 0.3 |
| | Thickness | |
| 6 | Standard for Comparison | 17.7 ± 0.1 |

As can be seen, the samples provided acceptable radiopacity in all tests and the branched grafts and most of the tantalum sheets provided excellent radiopacity results.

### Set #5

TABLE 25

| Sample # | Product Description |
|---|---|
| 1 | Gold wire 0.003 × 8 inches sealed graft |
| 2 | Running 3-0 tantalum suture on a 10 mm graft |
| 3 | Knotted multiple 3-0 tantalum sutures on a 10 mm graft |
| 4 | Loop of 3-0 tantalum suture with a knot |
| 5 | Sputter coated platinum braid |

A camera image of Set #5 is shown in FIG. 23A (aluminum step wedges and lead ingot shown in the center) and the raw x-ray image of Set #5 is shown in FIG. 23B. Set #5 tested some of the same samples that were included in Set #1, but a longer exposure time (2.30 mAs) was used in attempt to detect the least radiopaque sample. No equivalent aluminum thickness was provided for these samples, as this testing was used to provide a qualitative result of improved visibility of the samples under longer x-ray exposure time.

### Calibration Data for Determination of Equivalent Aluminum Thickness

TABLE 26

Set #1 10 mm mimic

| | Step Thickness | Step | Background | Difference |
|---|---|---|---|---|
| Wedge | (mm) | (px) | (px) | (px) |
| 3 mm | 3.0 | 209 | 255 | 46 |
| steps | 5.9 | 147 | 255 | 108 |
| | 9.0 | 102 | 255 | 0153 |
| | 12.0 | 71 | 255 | 184 |
| | 15.0 | 50 | 255 | 205 |
| | 18.0 | 34 | 255 | 221 |
| | 21.0 | 22 | 255 | 233 |
| | 24.0 | 15 | 255 | 240 |
| | 27.0 | 11 | 255 | 244 |
| | 30.1 | 12 | 255 | 243 |

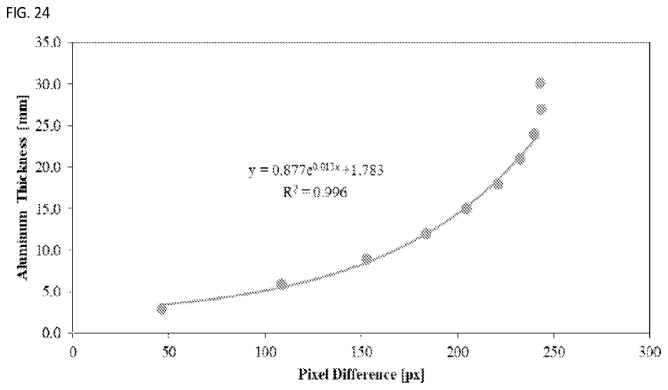

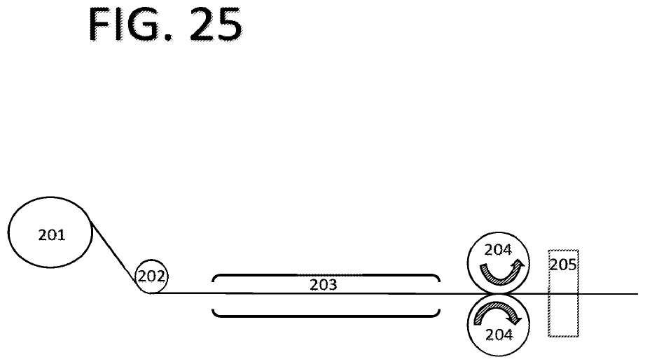

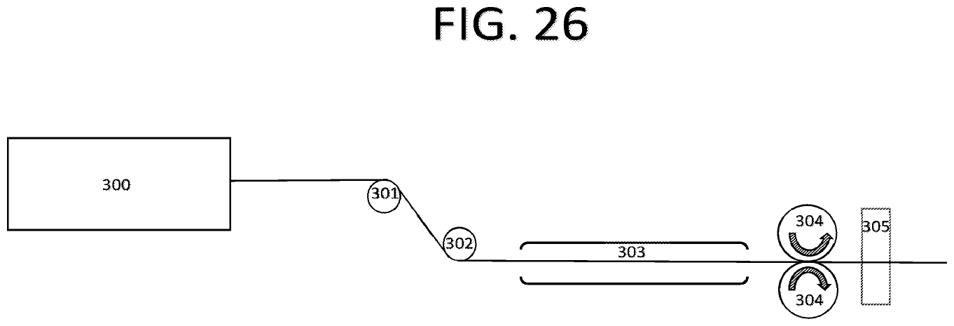

The calibration curve used for determination of the equivalent aluminum thickness for sample Set #1 is shown in FIG. 24. Step thicknesses 27.0 and 30.1 were not included in the calibration range due to near saturation. Similar processes and calibration curves were determined for Sets #2-4.

### Example 10

In this example, a radiopacity analysis was again conducted per ASTM F640-23. Samples were mounted onto 4″ by 6″ cardstock sheets in various groups. Each paper card containing samples was placed on the detector of a Faxitron Ultrafocus digital x-ray system, along with aluminum step wedges (alloy 1100, 0.5 mm and 3 mm steps) and a 10 mm aluminum body mimic (placed underneath the paper card). The samples were imaged under the following x-ray settings: source target-tungsten (W), filter type—none, detector type—CMOS, source to detector distance—24.57 inches, object to detector distance—0 inches, imaging system resolution—3072×2048 pixels, peak voltage—70 kV, exposure—1.44 mAs. Digital images were collected and analyzed by measuring pixel intensity of the sample and background sample areas. Pixel intensity measurements of the samples were taken at three different regions. Background pixel intensity measurements were taken at the background position nearest to the related sample portion. Pixel intensity differences for the aluminum stepped wedges were calculated in the same manner and were used to generate an exponential calibration curve. Equivalent aluminum thickness for the samples was then calculated using the aluminum calibration curve. Three individual readings per sample were taken with three controls (background measurements) immediate adjacent to the sample image. The three replicates provide the mean and standard deviation reported here.

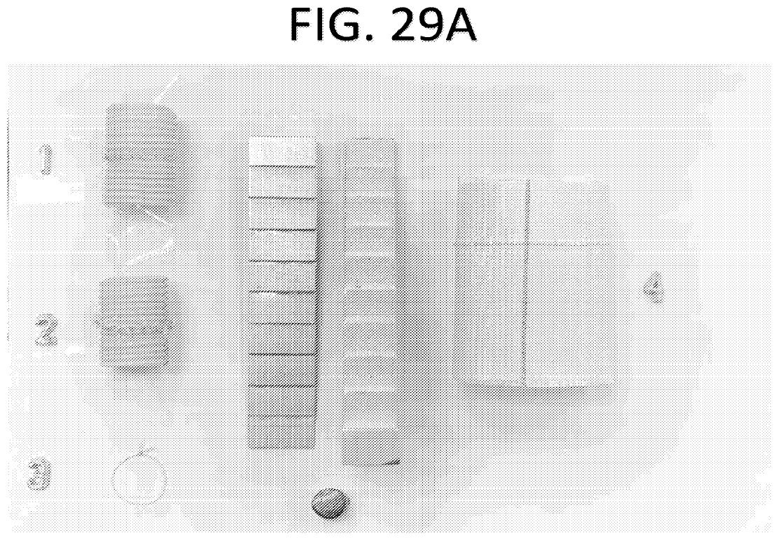

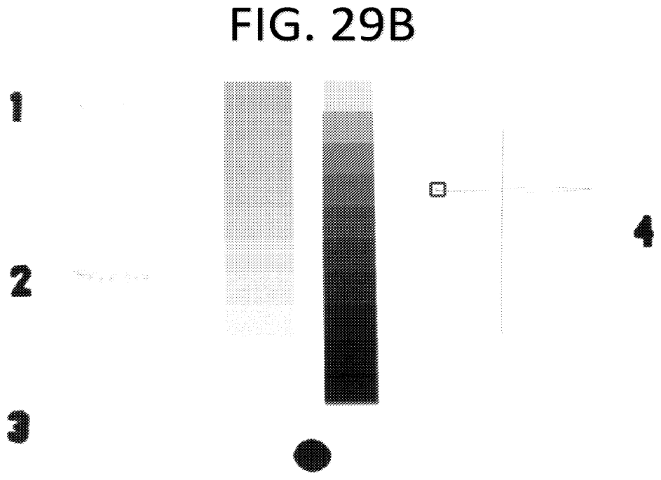

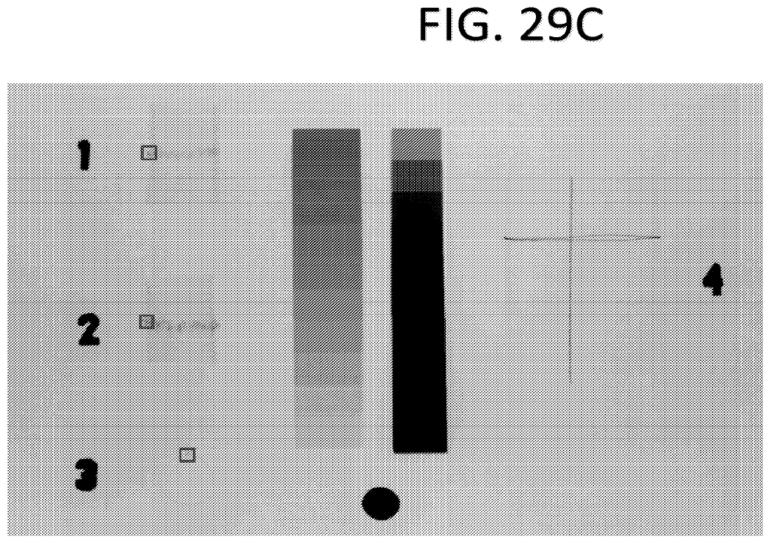

A camera image of the sample set is shown in FIG. 29A (aluminum step wedges and lead ingot shown in the center), the raw x-ray image is shown in FIG. 29B, and the contrast adjusted x-ray image is shown in FIG. 29C. Samples 1 and 2 were only slightly visible and sample 3 was not visible in the raw x-ray image. After adjustment of the window and level of the image, more detail could be observed in these samples in FIG. 29C. Samples 1 through 3 fell within the range of the 5 mm aluminum step wedge and sample 4 fell within the range of the 30 mm step wedge.

TABLE 27

| | | Equivalent |
|---|---|---|
| Sample | | Aluminum |
| # | Product Description | Thickness (mm) |
| 1 | 10 mm sealed graft with 5-0 ultra high | 2.2 ± 0.4 |
| | molecular weight polyethylene RO suture | |
| | running stitch | |
| 2 | 10 mm sealed graft with 5-0 ultra high | 2.2 ± 0.4 |
| | molecular weight polyethylene RO suture | |
| | knotted stitch | |
| 3 | 5-0 ultra high molecular weight polyethylene | 1.2 ± 0.1 |
| | RO suture tied in a loop | |
| 4 | 5-0 RO suture woven in weft and warp | 11.1 ± 0.8 |
| | direction on a graft | |

As can be seen, each experimental tantalum sample provided an equivalent aluminum thickness of between 1.2 and 11.1 mm. An equivalent aluminum thickness of above 10 mm is acceptable for arterial or endoscopic placement. As such, sample 4 was found to be acceptable.

### Calibration Data for Determination of Equivalent Aluminum Thickness.

TABLE 28

| | | 10 mm mimic | | |
|---|---|---|---|---|
| | Step Thickness | Step | Background | Difference |
| Wedge | (mm) | (px) | (px) | (px) |
| 0.5 mm steps | 0.5 | 160 | 177 | 16 |
| (contrast- | 1.0 | 146 | 177 | 31 |
| adjusted image | 1.6 | 130 | 179 | 49 |
| samples 1-3) | 2.1 | 118 | 179 | 61 |
| | 2.5 | 107 | 180 | 72 |
| | 3.1 | 94 | 181 | 86 |
| | 3.6 | 83 | 181 | 97 |
| | 4.1 | 74 | 181 | 108 |
| | 4.6 | 67 | 183 | 116 |
| | 5.0 | 64 | 186 | 123 |
| 3 mm steps | 2.9 | 222 | 255 | 33 |
| (raw image | 5.9 | 156 | 255 | 99 |
| sample 4) | 9.0 | 109 | 255 | 146 |
| | 12.0 | 78 | 255 | 177 |
| | 15.0 | 54 | 255 | 201 |
| | 18.0 | 37 | 255 | 218 |
| | 21.0 | 25 | 255 | 230 |
| | 24.0 | 17 | 255 | 238 |
| | 27.0 | 11 | 255 | 244 |
| | 30.1 | 13 | 255 | 242 |

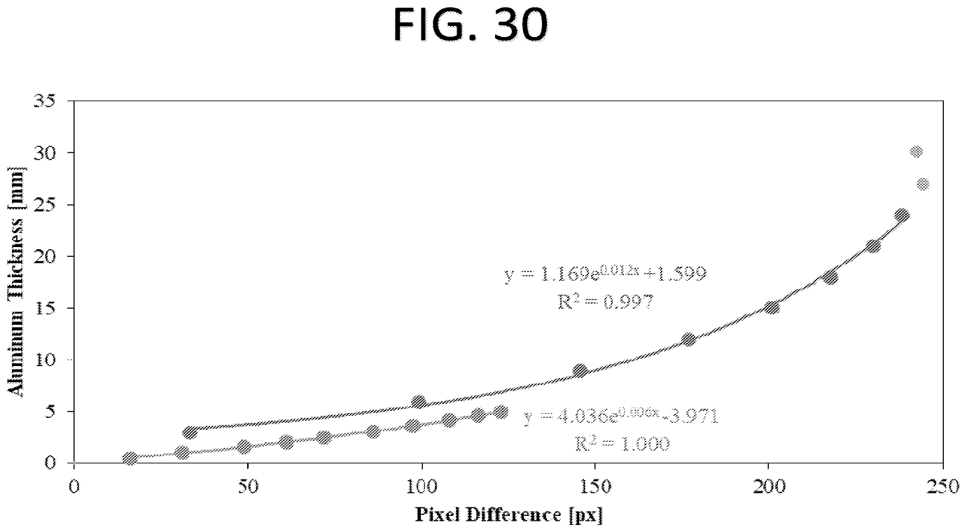

The calibration curve used for determination of the equivalent aluminum thickness is shown in FIG. 30. Step thicknesses 27.0 and 30.1 were not included in the calibration range due to near saturation.

### CONCLUSION

Many modification and other embodiments of the inventions set forth herein will come to mind to one skilled in the art to which these inventions pertain having the benefit of the teaching presented in the foregoing description and the associated drawings. Therefore, it is to be understood that the embodiments of the invention are not to be limited to the specific embodiments disclosed and that the modification and other embodiments are intended to be included within the scope of the invention. Moreover, although the foregoing descriptions and the associated drawings describe example embodiments in the context of certain example combinations of elements and/or functions may be provided by alternative embodiments without departing from the scope of invention. In this regard, for example, different combinations of elements and/or functions than those explicitly described above are also contemplated within the scope of the invention. As merely one example, any embodiment described as a ribbon may be utilized with a rod, a suture, or any other configuration. Although specific terms are employed herein, they are used in a generic and descriptive sense only and not for purposes of limitation.

1. A radiopaque component for in vivo medical use, the radiopaque component comprising a plurality of radiopaque particles entrapped within a biostable and biocompatible binder, wherein the radiopaque particles comprise at least 50%, by weight, of the radiopaque component.

2. The radiopaque component of claim 1, wherein the radiopaque particles comprise tantalum.

3. (canceled)

4. (canceled)

5. The radiopaque component of claim 1, wherein the binder is silicone.

6. The radiopaque component of claim 1, wherein the radiopaque component is conformable.

7. The radiopaque component of claim 1, wherein the radiopaque component has an equivalent aluminum thickness of at least 8 mm under x-ray.

8. (canceled)

9. (canceled)

10. The radiopaque component of claim 1, wherein the radiopaque component has a thickness of less than 0.6 mm.

11. The radiopaque component of claim 1, wherein the radiopaque component has a thickness of less than 0.2 mm.

12. The radiopaque component of claim 1, wherein the radiopaque particles comprise at least 60%, by weight, of the radiopaque component.

13. The radiopaque component of claim 1, wherein the radiopaque particles comprise at least 80%, by weight, of the radiopaque component.

14. The radiopaque component of claim 1, wherein the radiopaque particles comprise at least 90%, by weight, of the radiopaque component.

15. The radiopaque component of claim 1, further comprising a polymer affixed to the radiopaque component.

16. The radiopaque component claim 1, wherein the radiopaque component comprises a core and a polymer is braided, wrapped, or encased about the radiopaque component core to form a radiopaque suture.

17. (canceled)

18. (canceled)

19. The radiopaque component of claim 15, wherein the polymer comprises a core and the radiopaque component is braided, wrapped, or encased about the polymer core to form a suture.

20. The radiopaque component of claim 15, wherein the polymer comprises ultra high molecular weight polyethylene.

21. (canceled)

22. (canceled)

23. A radiopaque implantable medical device comprising:
an implantable medical device; and
a radiopaque component attached to the implantable medical device, the radiopaque component comprising a plurality of radiopaque particles entrapped within a biostable and biocompatible binder, wherein the radiopaque particles comprise at least 50%, by weight, of the radiopaque component.

24. The radiopaque implantable medical device of claim 23, wherein the radiopaque particles comprise tantalum and the radiopaque component is attached to the implantable medical device via silicone.

25. (canceled)

26. (canceled)

27. A radiopaque suture for in vivo use, the radiopaque suture comprising:
a radiopaque core, the radiopaque core comprising a plurality of radiopaque particles entrapped within a biostable and biocompatible binder, wherein the radiopaque particles comprise at least 50%, by weight, of the radiopaque core;
a polymer braided, wrapped, or encased about the radiopaque core to form a radiopaque suture.

28.-31. (canceled)

32. The radiopaque component of claim 16, wherein the radiopaque suture has a diameter within a range of 0.03 mm to 0.6 mm.

33. The radiopaque component of claim 15, wherein the polymer comprises polyester.

34. The radiopaque implantable medical device of claim 23, wherein the radiopaque component is woven and/or sewn into the implantable medical device.
